(19) United States
(12) Patent Application Publication
(10) Pub. No.: US 2003/0095965 A1
(43) Pub. Date: May 22, 2003

(54) Antibodies to Ly49E and CD94/NKG2 receptors

(76) Inventors: Katrien Van Beneden, Merelbeke (BE); Jozef De Boever, Lovendegem (BE); Georges Leclercq, Gent (BE)

Correspondence Address: NIXON & VANDERHYE, PC, 1100 N GLEBE ROAD, 8TH FLOOR, ARLINGTON, VA, 22201-4714, US

(21) Appl. No.: 10/136,436

(22) Filed: May 2, 2002

Related U.S. Application Data
(60) Provisional application No. 60/287,733, filed on May 2, 2001.

(51) Int. Cl. A61K 39/395; C07K 16/44; C07K 16/28

(52) U.S. Cl. 424/141.1; 424/144.1; 530/388.22; 530/387.3

(57) ABSTRACT

The present invention relates to monoclonal antibodies to the Ly49E and Ly49C receptors and to monoclonal antibodies to the CD94/NKG2A, CD94/NKG2C and/or CD94/NKG2E receptors, and to uses thereof in preventing, alleviating and/or treating diseases associated with transplantation (organ/bone marrow) or with autoimmunity.

The present application claims benefit of U.S. Provisional Application No. 60/287,733, filed May 2, 2001, the entire contents of which is incorporated herein by reference.

### FIELD OF THE INVENTION

The present invention relates to the field of antibodies to receptors on Natural Killer (NK) cells and their use in medical applications.

### BACKGROUND OF THE INVENTION

Natural killer cells are capable of lysing tumor and viral-infected cells that lack Major Histocompatibility Comples (MHC) Antigens (1, 2). NK cells also mediate “hybrid resistance” in which MHC-homozygous parental bone marrow grafts are rejected by F1 hybrids (3, 4). The discovery and cloning of receptors that recognize polymorphic MHC class I molecules and transduce inhibitory signals to NK cells revealed a molecular basis for the function of NK cells.

NK cell receptors can be divided into three families. One family contains the killer cell inhibitory receptors (KIR) (3) expressed on human NK cells (5). The second family consists of lectin-like CD94/NKG2A-F heterodimeric receptors that are expressed in humans and rodents (5-9). The third family has been identified in mice and contains the Ly49 receptors.

In C57BL/6 mice, the Ly49 family consists of 14 highly related genes (Ly49AN) encoding lectin-like glycoproteins expressed as disulfide-linked homodimers (10-14). The inhibitory receptors of the distinct families contain an immunoreceptor tyrosine-based inhibitory motif (ITIM) sequence in their cytoplasmic domain. Phosphorylation of tyrosine in the ITIM sequence recruits the intracellular protein phosphatase SHP-1 and eventually results in inhibition of NK cell activity (15-18). CD94/NKG2C, CD94/NKG2D, Ly49D and Ly49H, and KIR2DS NK cell receptors lack ITIM sequences in their cytoplasmic tail. These receptors are activating rather than inhibiting because they associate with adaptor proteins, DAP10 or DAP12, which transmit activating signals to NK cells (19-23).

The human and murine CD94/NKG2 heterodimers have been implicated in the recognition of the nonclassical MHC class I molecules, HLA-E for the human receptor and Qa-1^{b} for the mouse receptor (9, 24-27). Similar to HLA-E, the murine homologue Qa-1b binds TAP-dependent peptides derived from MHC class I signal sequences (28). It has recently been shown, using either Qa-1^{b} tetramers or an antiNKG2 mAb, that 50% of adult murine NK cells express CD94/NKG2 receptors (9, 27, 29). These CD94/NKG2⁺NK cells do not lyse target cells expressing Qa-1b (27, 30). Similar to human KIRs, murine Ly49 receptors have been shown to recognize different classical MHC class I molecules (5, 31-33). Inhibitory Ly49 receptors are expressed on subsets of NK cells that partially overlap (12, 34-37). Because Ly49A NK cells are present in B6 (H-2^{b}) mice that do not express the MHC class I ligand of Ly49A (H-2^{d}), the Ly49A NK cells in B6 mice would be auto aggressive (38-40). Therefore, it is accepted that each NK cell should express at least one inhibitory receptor that recognizes a self-MHC class I molecule to maintain self-tolerance (39, 40).

NK cells play a major role in immune responses to invading microorganisms, particularly viruses and intracellular pathogens. Human and mouse cytomegaloviruses contain open reading frames encoding structures similar to MHC class I that can bind NK cell receptors, thereby inhibiting NK cells and enhancing viral replication; the viral structures appear to be promiscuous, highlighting the significant contribution of NK cells in innate immunity to viruses and to other intracellular pathogens (5).

NK cells are involved in the rejection of both allogeneic bone marrow grafts and homozygous parental bone marrow grafts in heterozygous FI. recipients (known as the hybrid histocompatibility phenomenon) (42). The presence of NK cells in the F1 host recognize and eliminate the parental bone marrow calls as foreign because a subset of NK cells in the recipient lacks inhibitory receptors for the parental H2antigens. Further, NK cells appear to regulate autoimmunity, for example, in the development of experimental autoimmune encephalomyelitis.

NK Cell inhibitory receptors generally function in an inhibitory capacity, effectively slowing or even stopping NK cell activity. As a result, the use of specific molecules or antibodies which recognize these receptors may be useful to prevent these cells from binding to their natural ligand thereby impairing their natural cytolytic activity.

### SUMMARY OF THE INVENTION

The present invention relates to monoclonal antibodies which specifically bind to the Ly49E and Ly49C receptors and to monoclonal antibodies which specifically bind to the CD94/NKG2A, CD94/NKG2C and/or CD94/NKG2E receptors and/or fragments of these receptors. These receptors belong to two families of receptors that are present on Natural Killer (NK) cells: the CD94/NKG2 family which are present in human and mouse, and the Ly49 family which is present in mouse and for which the human homologue is not identified yet, and they belong to the same structural form of the C-type (Ca-dependent)-lectin-like receptors, which are disulfide linked, dimeric type II integral membrane proteins. These receptors mediate in the functionality of Natural Killer cells such as, lysing tumor and viral-infected cells that lack MHC Antigens, hybrid resistance versus transplants etc.

The present invention further provides fragments of the antibodies which retain binding specificity to the receptor and/or receptor fragments. The receptors which are the subject of the present invention are present on several subpopulations of hematopoietic cells such as, but not limited to, natural killer cells, memory T cells, natural killer T cells, γδ (gamma/delta) T cells and αβ (alpha/beta) T cells.

The monoclonal antibodies of the present invention are exemplified by antibody 4D12, which specifically binds to the inhibitory receptors Ly49E and Ly49C. The present invention is further exemplified by monoclonal antibody 3S9, which specifically binds to the CD94/NKG2A, CD94/NKG2C and/or CD94/NKG2E receptors.

The present invention also relates to fragments and/or homologues of the monoclonal antibodies described herein and also to humanized and/or chimeric versions thereof. The monoclonal antibodies, fragments, homologues, humanized and/or chimeric antibodies of the present invention are further, optionally, detectably labeled.

The invention further relates to a pharmaceutical composition comprising a monoclonal antibody of the invention, or a fragment, homologue or humanized or chimeric version thereof, optionally together with at least one anti-inflammatory or immunosuppressive drug.

The present invention further relates methods for obtaining and isolating hybridomas producing the monoclonal antibodies of the present invention and to the hybridomas obtained by such processes. The invention also relates to a process for producing monoclonal antibodies to receptors belonging to the Ly49 or to the CD94/NKG2 family with involve the culturing of the hybridomas of the present invention, in a manner known in the art.

The present invention also relates to a method for the detection of receptors belonging to the Ly49 or to the CD94/NKG2 family which method involves contacting an antibody or fragment of the present invention with a cell or sample suspected of containing a receptor of the noted family under conditions which allow binding of antibodies or fragments to receptors and, optionally, detecting or measuring the presence of the bound antibody- or fragment-receptor complex or detecting or measuring a product of such binding.

The present invention further provides a kit for the detection of a receptor of the present invention or a molecule which agonizes or antagonizes the binding of a receptor of the present invention and the antibody or fragment of the present invention which specifically binds the receptor, the kit containing a monoclonal antibody or fragment of the present invention and, optionally, direction for the use of such a kit. In one embodiment, the kit of the present invention provides at least one of the antibodies of the invention, the fragments, homologues or the humanized or chimeric versions thereof.

According to yet another embodiment, the invention relates to a method for inhibiting or enhancing the cytolytic activity of cells expressing a receptor of the Ly49 or of the CD94/NKG2 family.

Further the invention relates to a method for treating or preventing cell, tissue or organ transplant rejection in a mammalian recipient, which includes administering an antibody or antibody fragment of the Ly49 or of the CD94/NKG2 family to a recipient in need thereof, optionally further involving administering at least one anti-inflammatory or immunosuppressive drug prior to, with or subsequent to administering the noted antibody or antibody fragment.

The invention also relates to a method for treating or preventing autoimmune disease comprising administering an effective immunosuppressive amount of an antibody of the Ly49 family. Preferably said method of the invention uses an antibody against an inhibitory receptor of the Ly49 family, such as the Ly4E receptor or the Ly49C receptor. In a specifically exemplified embodiment of the present invention the antibody 4D12 may be used.

The invention further relates to a method for treating or preventing autoimmune disease comprising administering an effective immunosuppressive amount of an antibody of the CD94/NKG2 family. Preferably said method of the invention uses an antibody against an inhibitory receptor of the CD94/NKG2 family, such as the CD94/NKG2A receptor. In a specifically exemplified embodiment of the present invention the antibody 3S9 may be used.

The invention also relates to the use of an antibody of the invention, a fragment, a homologue or a humanized or chimeric version thereof as a medicament for preventing, treating or alleviating diseases or disorders associated with transplantation or with autoimmunity.

### LIST OF FIGURES

FIG. 1. Specificity of mAbs 4D12 and 3S9.

(A) HEK-T cells were transiently transfected with different Ly49 cDNAs, stained with FITC-conjugated mAb 4D12 and analyzed by flow cytometry (black histograms). HEK-T cells transfected with the hIL-2Rα cDNA in pcDNA1.1 were used as a negative control (open histograms). (B) HEK-T cells transiently transfected with CD94, NKG2A, CD94/NKG2A, CD94/DAP12/NKG2C or CD94/DAP12/NKG2E-HA cDNAs were stained with FITC-conjugated mAb 3S9 (black histograms). HEK-T cells transfected with the empty BSRαEN vector was used as a negative control (open histograms).

FIG. 2. Biochemical Analysis of Ly49E and NKG2A.

(A) IL-2-cultured FD17 thymocytes (lane 1), HEK-T cells transiently transfected with Ly49E cDNA in the expression vector BSRαEN (lane 2), and HEK-T cells transfected with the empty vector (lane 3) were surface biotinylated and lysed as described in Materials and Methods. Lysates were immunoprecipitated with mAb 4D12 (anti-Ly49E/C), separated by SDS-PAGE under non-reducing conditions, blotted, and incubated with streptavidin-conjugated POD (revealed with POD-precipitating substrate). (B) Ly49E-transfected HEK-T cells were surface biotinylated, lysed, and immunoprecipitated with mAb 4D12 (anti-Ly49E/C). Immunopreciprtations were treated under reducing conditions, in the absence (−) or presence (+) of N-Glycosidase F, separated by SDS-PAGE, and blotted as described in (A). (C) HEK-T cells transiently co-transfected with the expression vectors BSRαEN encoding CD94 cDNA and NKG2A cDNA (lane 2) and HEK-T cells transfected with the empty vector BSRαEN (lane 1) were surface biotinylated, lysed and immunoprecipitated with 3S9 mAb (anti-NKG2). Immunoprecipitations were separated by SDS-PAGE under reducing conditions, and blots were analyzed as described in (A).

FIG. 3. Tyrosine phosphorylation of Ly49E.

10-20×10⁶ IL-2-cultured FD17 thymocytes were either unstimulated (−) or stimulated (+) with pervanadate, lysed, and immunoprecipitated with mAb 4D12. Immunoprecipitations were separated by SDS-PAGE under non-reducing conditions; blots were incubated with POD-conjugated PY99 mAb (anti-phosphotyrosine) and revealed with POD-precipitating substrate.

FIG. 4. Expression of Ly49E on Fetal and Adult NK Cells.

(A) FD17 thymocytes, FD17 splenocytes, and adult splenocytes were freshly isolated and labeled with APC-conjugated anti-CD3, PE-conjugated anti-NK1.1, and FITC-conjugated mAb 4D12 (anti-Ly49E/C) or with FITC-conjugated anti-CD3, PE-conjugated anti-NK1.1, and biotin-conjugated mAb 4LO3311 (anti-Ly49C), revealed with streptavidin-APC, and analyzed by flow cytometry. Black histograms show staining of mAbs 4D12 or 4LO3311 on CD3-NK1.1⁺ gated cells. Open histograms represent staining with control mAbs. (B) Freshly isolated splenocytes from adult mice were stained with PerCP-conjugated anti-CD3, PE-conjugated anti-NK1.1, FITC-conjugated 4D12 and biotin-conjugated 4LO3311, revealed with streptavidin-APC or with PerCP-conjugated anti-CD3, biotin-conjugated anti-NK1.1, revealed with streptavidin-APC, FITC-conjugated 4D12 and PE-conjugated 5E6 (anti-Ly49C/I) and analyzed by gating on CD3⁻NK1.1 ⁺cells. (C) Freshly isolated splenocytes from adult mice were depleted of B cells. Cells were stained with FITC-conjugated anti-CD3, PE-conjugated 5E6, and biotin-conjugated 4LO3311 revealed with streptavidin-APC and analyzed by gating on CD3 cells. Results shown are representative of more than three experiments.

FIG. 5. Expression of CD94/NKG2, and Co-Expression with Ly49E on NK Cells from FD17 Thymocytes, FD17 Splenocytes, and Adult Splenocytes.

(A) Freshly isolated and IL-2-cultured cells were labeled with APC-conjugated anti-CD3, PE-conjugated ant-NK1.1, FITC-conjugated anti-NKG2A/C/E (mAb 3S9) and analyzed by flow cytometry. Black histograms show the expression of NKG2 on CD3⁻NK1.1⁺ gated cells. Open histograms represent staining with control rat mAb (IgG2b). (B) Freshly isolated and IL-2-cultured fetal thymocytes (left dot plots) and fetal splenocytes (right dot plots) were labeled with PerCP-conjugated anti-CD3, PE-conjugated anti-NK1.1, FITC-conjugated anti-NKG2 (mAb 3S9), biotin-conjugated anti-Ly49E/C (mAb 4D12), and revealed with streptavidin-APC. FCA shows co-expression of Ly49E/C and CD94/NKG2 on CD3⁻NK1.1⁺gated cells.

Results shown are representative of more than three experiments.

FIG. 6. Semi-Quantitative Analysis of NKG2A and CD94 in 3S9^{low} and 3S₉^{high} NK Cell Populations.

CD3⁻NK1.1⁺ cells from freshly isolated adult splenocytes were sorted into 3S9^{low} and 3S₉^{high} populations using mAb 3S9 (anti-NKG2). As a negative control, naive T cells (CD3⁺CD44⁻3S9⁻) cells were sorted from freshly isolated B6 lymph nodes. The purity of the sorted cells was ≧99.7%. cDNA was prepared and semi-quantitative RT-PCR for HPRT, NKG2A, and CD94 was performed. For semi-quantitative RT-PCR, four three-fold dilutions of each cDNA were amplified. H₂O and genomic DNA were used as negative controls (not shown).

FIG. 7. Frequency of NK Cells Expressing Ly49E and CD94/NKG2 During Ontogeny.

Splenocytes from mice of different ages were freshly isolated and incubated with APC-conjugated anti-CD3, PE-conjugated anti-NK1.1, FITC-conjugated 3S9 or with FITC-conjugated CD3, PE-conjugated anti-NK1.1, biotin-conjugated A1, biotin-conjugated 5E6, biotin-conjugated 4D11, revealed with streptavidin-APC or with PerCP-conjugated anti-CD3, PE-conjugated anti-NK1.1, FITC-conjugated 4D12, and biotin-conjugated 4LO3311, revealed with streptavidin-APC and analyzed on CD3⁻ NK1.1⁺ cells. To analyze the percentage of NK cells expressing Ly49E but not Ly49C, CD3⁻NK1.1⁺ cells were gated on the 4LO3311 population.

FIG. 8. Expression of Ly49E and CD94/NKG2 on Memory T cells and NK T Cells.

For analysis of memory T cells (upper histograms), lymph nodes from 11 month-old mice were freshly isolated. Cells were incubated with PerCP-conjugated anti-CD3, PE-conjugated anti-CD44, FITC-conjugated 3S9 (anti-NKG2), or with FITC-conjugated 4D12 (anti-Ly49E/C) and biotin-conjugated mAb 4LO3311 (anti-Ly49C), revealed with streptavidin-APC. Expression of Ly49E and CD94/NKG2 on memory T cells was analyzed by gating on respectively 4LO3311⁻D3⁺CD₄₄^{high} and CD3⁺CD₄₄high cells. For analysis of NK T cells (lower histograms), splenocytes from 2-month-old mice were freshly isolated. Cells were incubated with PerCP-conjugated anti-CD3, PE-conjugated anti-NK1.1, FITC-conjugated 4D12, and biotin-conjugated 4LO3311, revealed with streptavidin-APC or with APC-conjugated anti-CD3, PE-conjugated anti-NK1.1, and FITC-conjugated 3S9. Expression of Ly49E and CD94/NKG2 on NK T cells was analyzed by gating on respectively CD3^{int}NK1.1⁺4LO3311⁻ and CD3^{int}NK1.1⁺ splenocytes.

FIG. 9. Cytotoxicity of IL-2-Cultured Ly49E⁺CD94/NKG2^{low} and Ly49E⁻CD94/NKG2^{high} Fetal NK Cells.

(A) After culture of FD17 thymocytes with IL-2 for 4 days, CD3⁻NK1.1⁺cells were sorted into either Ly49E⁺CD94/NKG2^{low} and Ly49E⁻CD94/NKG2^{high} subsets using mAbs 4D12 (anti-Ly49E/C) and 3S9 (anti-NKG2) as shown in the regions of the dot plot (B) After 3 days of additional IL-2 culture, the cytotoxicity of Ly49E⁺CD94/NKG2^{low} (diamonds) and Ly49E⁻CD94/NKG2^{high} (squares) fetal NK cells was analyzed in a ⁵¹Cr release assay against RMA (open symbols) and RMA-S (black symbols) tumor cells.

FIG. 10. Expression LEVELS of Ly49E and CD94/NKG2 on NK Cells from B6 WT and β₂m⁻1 Mice.

FD17 thymocytes were freshly isolated and stained with APC-conjugated anti-CD3, PE-conjugated anti-NK1.1, and FITC-conjugated 4D12 or with FITC-conjugated 3S9. Histograms show the median fluorescence intensity (MFI) of Ly49E (upper histograms) and CD94/NKG2 (lower histograms) by gating on CD3⁻NK1.1⁺ cells.

FIG. 11. Ly49E/C and NKG2 Expression by TCR Vγ3 Cells During Differentiation in the Fetal Thymus.

FD15 to FD18 B6 thymocyles were freshly isolated and stained with the following mAbs: PE-conjugated anti-Vγ3, biotin-conjugated anti-HSA, revealed with streptavidin-APC, and FITC-conjugated 4D12 or FITC-conjugated 3S9. Expression was analyzed by flow cytometry by gating on the Vγ3⁺cells. Expression of Ly49E/C or NKG2A/C/E on HSA^{low}Vγ3⁺ T cells is shown between parenthesis. Results shown are representative of three experiments.

FIG. 12. Expression of NK Receptors on Fetal Thymic and Skin-Located Vγ3⁺ T Cells.

Freshly isolated FD 17 B6 thymocytes and epidermal cells prepared from adult B6 mice were stained with PE- or FITC-conjugated anti-Vγ3 mAb in combination with FITC-conjugated 3S9, FITC-conjugated 4D12, biotin-conjugated 12A8, revealed with streptavidin-PE, PE-conjugated 5E6, FITC-conjugated 4D11, PE-conjugated anti-2B4, or FITC-conjugated anti-IL-2Rβ mAbs. Empty histograms show the expression of indicated receptors by gating on Vγ3⁺ T cells. Black histograms represent background staining. Results shown are representative of more than three experiments.

FIG. 13. Ly49E/C and CD94/NKG2 Expression by Non-Vγ3 γδ T Cells.

(A) Freshly isolated neonatal (day 0) B6 thymocytes were depleted for CD4⁺T cells and stained with following mAbs: FITC-conjugated 3B9, FITC-conjugated 4D12, or biotin-conjugated 4LO3311, and PE- or FITC-conjugated anti-Vγ3, and PE- or biotin-conjugated anti-γδ TCR, revealed with streptavidin-APC. Expression of NK receptors on γδ T cells was analyzed by gating on Vγ3⁻ thymocytes. Freshly isolated splenocytes from adult mice were depleted for CD4⁺ T cells and B cells. Cells were stained with FITC-conjugated 3S9, FITC-conjugated 4D12, or biotin-conjugated 4LO3311, and PE- or biotin-conjugated anti-γδ TCR, revealed with streptavidin-APC. The percentages shown in the upper right quadrant of the dot plots represent the expression of the indicated NK receptor on γδ T cells. (B) Dot plot shows co-staining with mAbs 4D12 and 4LO3311 by gating on TCR γδ⁺ splenocytes. Results are representative of three experiments.

FIG. 14. Vγ3⁺ Thymocytes Expressing NK Receptors Exhibit a Memory Phenotype.

FD17 B6 thymocytes were freshly isolated and stained with the following mAbs: FITC-conjugated 4D12 (anti-Ly49E/C), FITC-conjugated 3S9 (anti-NKG2A/C/E), or FITC-conjugated anti-1L-2Rβ (CD122) in combination with biotin-conjugated anti-HSA, or biotin-conjugated anti-IL-2Rα (CD25), revealed with streptavidin-APC and PE-conjugated anti-Vγ3 or in combination with PE-conjugated anti-CD44 and biotin-conjugated anti-Vγ3, revealed with streptavidin-APC. When PE-conjugated ant-2B4 was used, thymocytes were stained with biotin-conjugated anti-HSA, revealed with streptavidin-APC and FITC-conjugated anti-Vγ3. Cells were analyzed by flow cytometry by gating on Vγ3⁺cells. Results shown are representative of three experiments.

FIG. 15. Comparison of Ly49E and CD94/NKG2 Expression on Vγ3⁺ T Cells from FD17 WT and β₂m⁺ Mice.

FD17 thymocytes from WT and β₂m⁺ mice were freshly isolated and stained with FITC- or PE-conjugated anti-V@, biotin-conjugated anti-HSA and with FITC-conjugated 4D12, FITC-conjugated 3S9 mAbs, or PE-conjugated 5E6. FCA was performed by gating on Vγ3⁺ cells. Results shown are representative of three experiments.

FIG. 16. CD94/NKG2-Mediated Inhibition of Tumor Cell Lysis by Vγ3 T Cells.

After culture of FD17 thymocytes with r-IL-2 for 3 days, Vγ3⁺ T cells were sorted using FITC-conjugated anti-TCR Vγ3 mAb or using PE-conjugated anti-TCR Vγ3 mAb combined with FITC-conjugated anti-NKG2 (3S9) mAb. After 3 days of additional r-IL-2 culture, Vγ3⁺ T cells were used as effector cells in a ⁵¹Cr-release assay. Purity of sorted Vγ3⁺, Vγ3⁺ CD94/NKG2^{low}, and Vγ3⁺ CD94/NKG2^{high} T cells was >98%, 77%, and 96%, respectively. Cytotoxicity of (A) Vγ3⁺T cells and (B) Vγ3⁺ CD94/NKG2^{low} and Vγ3^{+ CD}94/NKG2^{high} T cells against Fcγ^{R+} P815 target cells in the presence of 30 μg/ml anti-NKG2 (3S9) or isotype control (IgG2b) mAbs. (C) Cytotoxic activity of Vγ3⁺ T cells against Qa1^{b}-transfected T2Q target cells pre-incubated with Qdm peptide or unrelated peptide, and against untransfected T2 cells incubated with Qdm peptide. In blocking studies, T2Q target cells loaded with Qdm peptide were incubated with anti-Qa1^{b} mAb. (D) Cytotoxicity of Vγ3⁻D94/NKG2^{low} and Vγ3⁺CD94/NKG2^{high} T cells against Qa1^{b}-transfected T2Q target cells pre-incubated with Qdm peptide or unrelated peptide.

### DETAILED DESCRIPTION OF THE INVENTION

Natural Killer cell receptors fall into two structural forms: (i) C-type (Ca dependent)-lectin-like receptors that are disulfide linked, dimeric type II integral membrane proteins including the human CD94 and NKG2 family (43) and the mouse Ly49 family (46); and (ii) immunoglobulin-like receptors which are usually monomeric, type I integral membrane proteins that include the human killer inhibitory receptors (KIR, p58 and p7O1pI4O) and the activating receptors (KAR) (5). Despite these structural differences, studies indicate that both types of receptors are polymorphic at several levels: the receptors belong to several families of highly conserved molecules that display allelic forms; many of these molecules are expressed on subsets of NK cells and an individual NK cell may express several different receptors; the inhibitory receptors of both structural groups mediate their effects through cytoplasmic sequences termed immunoreceptor tyrosine-based inhibitory motifs (ITIMs).

Most, but not all, of the Ly49 receptors contain an ITIM in their cytoplasmic domains, which upon tyrosine phosphorylation, recruits SH2-containing tyrosine phosphatase-1 (SHP-1) or SHP-2 that subsequently inhibits NK cell effector function.

The present invention provides a monoclonal antibody which generally, or preferably, specifically recognizes and binds the inhibitory receptor Ly49E of the Ly49 family of receptors, which may be in isolated form or may be present on a cell, such as, a hematopoietic cell, selected from, but not restricted to, any one of a natural killer cell, a memory T cell, a natural killer T cell, a gamma/delta T cell and an alpha/beta T cell and/or as a soluble or membrane bound component of a recombinant host cell.

The monoclonal antibodies of the invention prevent the binding of the receptors with their ligand, wherein the ligand may be an MHC class 1b molecule.

The present invention further provides a monoclonal antibody which generally, or specifically, recognizes and binds inhibitory receptors Ly49E and Ly49C of the Ly49 family of receptors, which may be in isolated form or may be present on a cell, such as, a hematopoietic cell, selected from, but not restricted to, any one of a natural killer cell, a memory T cell, a natural killer T cell an alpha/beta T cell and a gamma/delta T cell and/or as a soluble or membrane bound component of a recombinant host cell.

The present invention further provides a monoclonal antibody termed 4D12 which binds to the inhibitory receptors Ly49E and Ly49C of the Ly49 family.

The above-mentioned Ly49⁺ hematopoietic cells do not lyse target cells expressing MHC class Ib molecules. As most of the Ly49 receptors are inhibitory receptors and as the signal generated by inhibitory receptors is dominant compared to the signal generated by activating receptors, cross-linking of all Ly49 receptors with said monoclonal antibody, and preferably with bivalent 4D12, will result in an overall inhibition of NK cells and of cells expressing Ly49 receptors.

CD94 and NKG2 form a heterodimer composed of a CD94 glycoprotein that is disulfide-bonded to either an NKG2A, NKG2C or NKG2E subunit (5). CD94 lacks a cytoplasmic domain, but is required for transport and membrane expression of the NKG2A, NKG2C or NKG2E glycoproteins. Although similar in their extracellular regions, NKG2A possesses an ITIM in its cytoplasmic domain, whereas the NKG2C and NKG2E molecules lack an ITIM, but contain a basic amino acid in their transmembrane domains that may provide for association with other signaling/transducing proteins. Therefore, the heterodimer containing NKG2A is inhibiting, whereas the two other heterodimer are activating. It should be noted however that heterodimers with NKG2A represent ˜95% of the total number of NKG2 receptors in NK cells (88).

In a further embodiment, the present invention provides a monoclonal antibody which specifically recognizes and binds to the CD94/NKG2A, CD94/NKG2C and the CD94/NKG2E receptors of the CD94/NKG2 family, which may be isolated form or may be present on a cell, such as, a hematopoietic cell, selected from, but not restricted to, any one of a natural killer cell, a memory T cell, a natural killer T cell, an alpha/beta T cell and a gamma/delta T cell and/or as a soluble or membrane bound component of a recombinant host cell.

The present invention provides, as a specifically exemplified embodiment of the present invention, an antibody termed 3S9, which recognizes and binds to CD94/NKG2A, CD94/NKG2C and CD94/NKG2E receptors as described herein.

The monoclonal antibody of the present invention or fragment thereof may prevent the binding of the receptors with their ligand, wherein the ligand may be an MHC class 1b molecule. The ligand for human CD94/NKG2 heterodimer receptors has been identified as the nonclassical MHC class I molecule HLA-E. The above-mentioned CD94/NKG2⁺ hematopoietic cells (e.g. NK cells) do not lyse target cells expressing MHC class I HLA-E molecules. As heterodimers with NKG2A represent ˜95% of the total number of NKG2 receptors in NK cells (88), and as the signal generated by inhibitory receptors is dominant compared to the signal generated by activating receptors, cross-linking of all CD94/NKG2 receptors with said monoclonal antibody, and preferably with bivalent 3S9 antibody, will result in an overall inhibition of NK cells and of cells expressing CD94/NKG2 receptors.

Hematopoietic cells, as described herein, may be derived and/or isolated—from, for instance from blood, thymus, spleen, lymph nodes or other body fluids or organs containing hematopoietic or natural killer cells expressing Ly49 or CD94/NKG2 receptors

The antibodies directed against the receptors as indicated above may show utility as a issue or cell marker for the above listed cell types of fluids of tissues.

In a further embodiment, the present invention provides a fragment or homologue of any of the monoclonal antibodies described herein, wherein the fragment may be a monovalent Fab or divalent F(ab′)₂ fragment of the monoclonal antibodies and the noted homologue may be an antibody mimic.

Opposite to the effect of bivalent 3S9 antibody, monovalent Fab fragments of the 3S9 antibody will not result in cross-linking of the CD94/NKG2 receptors, but will prevent the receptors from binding their physiological ligand Qa1b (mouse) or HLA-E (human). This will result in the absence of inhibitory signals, in activation of the NK cells, and in killing of the target cells.

Similary, monovalent Fab fragments of the 4D12 antibody will prevent the inhibiting Ly49E and Ly49C receptor from binding their physiological ligand which will result in the absence of inhibitory signals, in activation of the NK cells, and in killing of the target cells.

The term “antibody”, as used herein, refers to immunoglobulin molecules and immunologically active portions of immunoglobulin molecules, i.e., molecules that contain an antigen-binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecules.

Antibodies of the invention include, but are not limited to, polyclonal, monoclonal, human, humanized or chimeric antibodies. Most preferably the antigen-binding antibody fragments of the present invention include, but are not limited to, Fab, Fab′ and F(ab′)2, Fd, single-chain Fvs (scFv), single-chain antibodies, disulfide-linked Fvs (sdFv) fragments and fragments comprising either a VL or VH domain, fragments produced by a Fab expression library, anti-idiotypic (anti-Id) antibodies (including, e.g., anti-id antibodies to antibodies of the invention), and epitope-binding fragments of any of the above. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce Fab′)2 fragments). F(ab′)2 fragments contain the variable region, the light chain constant region and the CH1 domain of the heavy chain.

Techniques to recombinantly produce Fab, Fab′ and F(ab′)2 fragments can also be employed using methods known in the art such as those disclosed in PCT publication WO 92/22324 and in (63), (66) and (67).

Examples of techniques which can be used to produce single-chain Fvs and antibodies include those described in U.S. Pat. Nos. 4,946,778 and 5,258,498 and further in (76), (77) and (78).

The antibodies of the invention may be from any animal origin including birds and mammals. Preferably, the antibodies are human, murine, rat, hamster, donkey, sheep, rabbit, goat, guinea pig, camel, horse, or chicken.

For some uses, including in vivo use of antibodies in humans and in vitro detection assays, it may be preferable to use chimeric, humanized, or human antibodies.

Therefore, according to a more specific embodiment the invention provides humanized or chimeric version derivable from any of the above-described antibodies. The present invention provides, as a specifically exemplified embodiment of the present invention a humanized or chimeric version of the antibody 4D12, as herein described The present invention further provides, as a specifically exemplified embodiment of the present invention a humanized or chimeric version of the antibody 3SG, as herein described.

A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art, see for instance in U.S. Pat. Nos. 5,807,715; 4,816,567; and 4,816,397 and further in (79), (80) and (81).

Humanized antibodies are antibody molecules from non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See for instance U.S. Pat. No. 5,585,089 and (82))

Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596 and further (83), (84) and (85)), and chainshuffling (U.S. Pat. No. 5,565,332).

Completely human antibodies, which recognize a selected epitope can be generated using a technique, referred to as “guided selection.” In this approach a selected non-human monoclonal antibody, e.g., a mouse antibody, is used to guide the selection of a completely human antibody recognizing the same epitope. (89).

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. (See, for instance, PCT publications WO 93/17715; WO 92/08802; WO 91/00360) The antibodies of the invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The present invention further provides the herein described antibodies, fragments, homologues, chimeric version or humanized version also containing at least one detectable label.

The present invention provides, as a specifically exemplified embodiment of the present invention, the antibody 3S9 or a humanized or chimeric version thereof, containing at least one detectable label. The labeled 3S9 antibody may be used in at least one of the methods herein described.

Antibodies of the present invention may also be described or specified in terms of their cross-reactivity. Antibodies (also termed homologous antibodies) that bind polypeptides with at least ˜95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. In specific embodiments, antibodies of the present invention cross-react with murine, rat and/or rabbit homologues of human proteins and the corresponding epitopes thereof.

The antibodies of the present invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in (86) and (87)

The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art and are discussed in detail in the Examples.

In a non-limiting example, mice can be immunized with a polypeptide of the invention or a cell expressing such peptide. Once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP2/0 available from the ATCC.

Hybridomas are selected and cloned by limited dilution or are selected by flow cytometry techniques as described in detail in the examples. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention.

According to a specific embodiment the invention provides a hybridoma producing a monoclonal antibody having a reactivity substantially identical or identical to that of least any of the monoclonal antibodies described herein.

In a further embodiment, the present invention provides a method for the detection of receptors belonging to the Ly49 or CD94/NKG2 family which includes an immunological binding or association of a monoclonal antibody, a fragment, a homologue, or a chimeric or a humanized version thereof as described herein, said method comprising contacting a monoclonal antibody of the invention, or a fragment, a homologue or a chimeric or a humanized version thereof with a cell suspected of expressing a receptor belonging to said family of receptors and detecting and/or measuring the binding or association of said antibody with said cell expressing said receptor. The detection and/or measuring of the method of the present invention may by performed, for example, by a fluorescent activated cell sorter (FACS), an enzyme linked immunosorbent assay (ELISA), a radioimmunoassay (RIA), a fluorescent immunoassay (FIA), an immune radio metric assay (IRMA), a chemiluminescent immuno assay (CLIA), an electro chemiluminescent immuno assay (ECL), an agglutination assay, a histological immuno assay as well as all possible Western blot analyses.

The present invention provides a kit for the detection of a receptor of the present invention belonging to the Ly49 or CD94/NKG2 family, which agonizes or antagonizes the binding of a receptor of the present invention and the antibody or fragment of the present invention which specifically binds the receptor, the kit containing a monoclonal antibody or a fragment, a homologue or a chimeric or humanized version thereof and optionally direction fro the use of such a kit. In one embodiment the kit of the present invention contains at least one of the antibodies of the invention, optionally buffers or ingredients useful in making buffers useful in detecting and/or measuring binding and/or association of the monoclonal antibody, fragment, homologue or chimeric or humanized version thereof, and further optionally, a detectable label and/or detectable second component to aid in detecting and/or measuring the binding between the antibody and said receptor or cell expressing said receptor.

The present invention further provides a pharmaceutical composition containing at least one monoclonal antibody as described herein, or a fragment, a homologue or a chimeric or humanized version thereof as described herein, optionally in admixture with a pharmaceutically acceptable carrier.

The present invention provides a process for obtaining and isolating a hybridoma of the present disclosure, said hybridoma secreting a monoclonal antibody described herein, comprising:

optionally, starting from the spleen cells of an animal, e.g. mouse, hamster or rat, previously immunized in vivo with an antigen expressing cell chosen from hematopoietic cells, thymocytes or splenocytes from rat or mice,

optionally, fusing said immunized cells with myeloma cells under hybridoma-forming conditions, and,

selecting those hybridomas which secrete monoclonal antibodies which specifically recognize receptors belonging to the Ly49 or/and CD94/NKG2 family.

The present invention provides monoclonal antibodies as described herein obtainable by a process of at least the following steps of:

optionally, starting from the spleen cells of an animal, e.g. mouse, hamster or rat, previously immunized in vivo with an antigen expressing cell chosen from hematopoietic cells, thymocytes or splenocytes from rat or mice,

optionally, fusing said immunized cells with myeloma cells under hybridoma-forming conditions,

selecting those hybridomas which secrete monoclonal antibodies which specifically recognize inhibitory receptors belonging to the Ly49 or/and CD94/NKG2 family,

culturing the selected hybridomas as described herein, or the hybridomas obtained through the process described above, in a suitable or appropriate culture medium,

recovering the monoclonal antibodies excreted by said selected hybridomas; or alternatively,

implanting the selected hybridomas described herein, or the hybridomas obtained through the process described above, into the peritoneum of a mouse and, when ascites has been produced by the animal, recovering the monoclonal antibodies then formed from said ascites.

The present invention provides a process for producing monoclonal antibodies as described herein comprising:

culturing the selected hybridomas obtained as described herein in an appropriate medium culture,

recovering the monoclonal antibodies excreted by said selected hybridomas; or alternatively,

implanting the selected hybridomas described herein, or the hybridomas obtained through the process as described above into the peritoneum of a mouse and, when ascites has been produced by the animal, recovering the monoclonal antibodies then formed from said ascites.

The present invention provides a method for inhibiting or enhancing the cytolytic activity of cells expressing a receptor of the Ly49 or CD94!NKG2 family comprising administering or adding a monoclonal antibody as described herein or a fragment, a homologue, or a humanized or chimeric version thereof as herein described, to a mammalian recipient or to a cell culture or tissue culture.

CD94 is coexpressed as a heterodimer with either NKG2A, NKG2C or NKG2E. The heterodimer containing NKG2A is inhibiting, whereas the two other heterodimers are activating. As heterodimers with NKG2A represent ˜95% of the total number of NKG2 receptors in NK cells (88), and as the signal generated by inhibitory receptors is dominant compared to the signal generated by activating receptors, cross-linking of all CD94/NKG2 receptors by bivalent 3S9 monoclonal antibody will result in an overall inhibition of NK cells. On the contrary, monovalent Fab fragments of the 3S9 antibody will not result in cross-linking of the CD94/NKG2 receptors, but will prevent the receptors from binding their physiological ligand Qa1b (mouse) or HLA-E (human). This will result in the absence of inhibitory signals, in activation of the NK cells, and in killing of the target cells. Bivalent 4D12 antibodies and monovalent Fab fragments of 4D12 will act similarly towards their receptor and towards the cells expressing their receptor as described for 3S9. Therefore, bivalent 4D12 may inhibit NK cells whereas monovalent Fab fragments of 4D12 may be used for killing target cells.

The invention provides methods of preventing acute rejection following bone marrow or solid organ transplantation. The methods entail administering, e.g. intravenously, to a transplant patient a monoclonal antibody which binds to a receptor of the Ly49 family present on human hematopoietic cells, such as T lymphocytes important for organ and bone marrow transplantation and natural killer cells for bone marrow transplantation. The methods further entail administering, e.g. intravenously, to a transplant patient a monoclonal antibody which binds to a receptor of the or CD94/NKG2 family present on human hematopoietic cells, such as T lymphocytes important for organ and bone marrow transplantation and natural killer cells for bone marrow transplantation. According to a specifically exemplified embodiment of the present invention, the antibody is 3S9 or 4D12.

The monoclonal antibody is preferably a chimeric or humanized antibody that blocks binding of human hematopoietic cells to the cells of the transplant. In some methods, a single dose of about 1 mg/kg of antibody is administered about every other week, commencing immediately prior to transplantation and continuing until 8 weeks after transplantation. According to a specifically exemplified embodiment of the present invention, the antibody is a chimeric or humanized antibody derived from 3S9 or derived from 4D12.

The invention further provides a method for treating or preventing cell, tissue or organ transplant rejection in a mammalian recipient, comprising administering an antibody that binds to an inhibitory receptor of the Ly49 family in an amount effective to inhibit an immune-mediated response in the recipient to said transplant, characterized that said antibody is an antibody described herein or a fragment, a homologue, or a humanized or chimeric version thereof. According to a specifically exemplified embodiment of the present invention, the antibody is 4D12. According to a further specifically exemplified embodiment of the present invention, the antibody is a chimeric or humanized antibody derived from 4D12.

The invention further provides a method for treating or preventing cell, tissue or organ transplant rejection in a mammalian recipient, comprising administering an antibody that binds to an inhibitory receptor of the or CD94/NKG2 family in an amount effective to inhibit an immune-mediated response in the recipient to said transplant, characterized that said antibody is an antibody described herein or a fragment, a homologue, or a humanized or chimeric version thereof. According to a specifically exemplified embodiment of the present invention, the antibody is 3S9. According to a further specifically exemplified embodiment of the present invention, the antibody is a chimeric or humanized antibody derived from 3S9.

The invention provides a method as described above wherein the amount is effective to induce immune tolerance in the recipient to the transplant.

The above methods may be applied in those cases wherein the cell, tissue or organ transplant is one of the following (but not limited to) a pancreatic islet, a kidney, a heart, a vascular tissue, a hematopoietic cell or a liver.

The invention provides to any of the above described methods wherein the recipient is human or an animal.

The invention more specifically provides a method for reducing the incidence of acute rejection episodes following transplantation, comprising administering to human patients undergoing transplantation a therapeutically effective dosage of a chimeric or humanized monoclonal antibody as described herein that binds to an inhibitory receptor of the Ly49 family. Said antibody may cross-link the Ly49 receptors of the natural killer cells, thereby preventing that these cells kill the target cell. Said antibody may also inhibit binding of the cell comprising said receptor to the cells of the transplant. According to a specifically exemplified embodiment of the present invention, the antibody is a 4D12.

The invention more specifically provides a method for reducing the incidence of acute rejection episodes following transplantation, comprising administering to human patients undergoing transplantation a therapeutically effective dosage of a chimeric or humanized monoclonal antibody as described herein that binds to an inhibitory receptor of the CD94/NKG2 family. Said antibody may cross-link the CD94/NKG2 receptors of the natural killer cells, thereby preventing that these cells kill the target cell. Said antibody may also inhibit binding of the cell comprising said receptor to the cells of the transplant According to a specifically exemplified embodiment of the present invention, the antibody is 3S9.

The invention further provides a method for treating an individual having a transplanted organ, tissue or cell, comprising administering a therapeutically effective amount of any of the humanized antibodies herein described.

The invention further provides a pharmaceutical composition comprising an effective immunosuppressive amount of an antibody which binds to an inhibitory receptor of the Ly49 family in combination with a pharmaceutically acceptable vehicle. According to a specifically exemplified embodiment of the present invention, the antibody is 4D12 or a chimeric or humanized antibody derived from 4D12, or a fragment or a homologue thereof.

The invention further provides a pharmaceutical composition comprising an effective immunosuppressive amount of an antibody which binds to an inhibitory receptor of the CD94/NKG2 family in combination with a pharmaceutically acceptable vehicle. According to a specifically exemplified embodiment of the present invention, the antibody is 3S9 or a chimeric or humanized antibody derived from 3S9, or a fragment or a homologue thereof.

The invention further relates to said pharmaceutical composition wherein the amount is effective to induce immune tolerance in a mammal recipient of an allogeneic or xenogeneic organ, tissue or cell transplant.

An allogeneic transplant is a transplant derived from a matched healthy donor, usually a brother or sister. The term xenogeneic is usually applied to tissue or cells from another species.

The invention further provides a method for treating or preventing cell, tissue or organ transplant rejection in a mammalian recipient further comprising administering at least one anti-inflammatory or immunosuppressive drug. Said anti-inflammatory or immunosuppressive drug may be chosen from cyclosporin, cyclophosphamide, FK-506, rapamycin, corticosteroids, mycophenolate mofetil, leflunomide, anti-lymphocyte globulin, deoxyspergualin or OKT.

The invention thus also provides a pharmaceutical composition comprising an effective immunosuppressive amount of an antibody which binds to an inhibitory receptor of the Ly49 family, said composition further comprising an anti-inflammatory or immunosuppressive drug. According to a specifically exemplified embodiment of the present invention, the antibody is 4D12, or a chimeric or humanized antibody derived from 4D12, or a fragment or a homologue thereof.

The invention thus also provides a pharmaceutical composition comprising an effective immunosuppressive amount of an antibody which binds to an inhibitory receptor of the CD94/NKG2 family, said composition further comprising an anti-inflammatory or immunosuppressive drug. According to a specifically exemplified embodiment of the present invention, the antibody is 3S9, or a chimeric or humanized antibody derived from 3S9, or a fragment or a homologue thereof.

For instance said pharmaceutical composition may be used in combination therapy.

Therefore the invention also provides a combination therapy for treating or preventing cell, tissue or organ transplant rejection in a mammalian recipient comprising administering an effective immunosuppressive amount of an antibody which binds to an inhibitory receptor of the Ly49 family and an anti-inflammatory or immunosuppressive drug. According to a specifically exemplified embodiment of the present invention, the antibody is 4D12 or a chimeric or humanized antibody derived from 4D12, or a fragment or a homologue thereof.

Therefore the invention also provides a combination therapy for treating or preventing cell, tissue or organ transplant rejection in a mammalian recipient comprising administering an effective immunosuppressive amount of an antibody which binds to an inhibitory receptor of the CD94/NKG2 family and an anti-inflammatory or immunosuppressive drug. According to a specifically exemplified embodiment of the present invention, the antibody is 3S9 a chimeric or humanized antibody derived from 3S9, or a fragment or a homologue thereof.

The antibodies of the invention are also useful In treating, preventing or alleviating diseases associated with autoimmunity, for instance inflammatory bowel disease, multiple sclerosis, Type I diabetes, systemic lupus erythematosus or rheumatoid arthritis

The invention thus provides a method for treating or preventing autoimmune disease comprising administering an effective immunosuppressive amount of an antibody which binds to an inhibitory receptor of the Ly49 or of the CD94/NKG2 family. Preferably, an inhibitory receptor of the Ly49 or of the CD94/NKG2 family is envisaged. According to a specifically exemplified embodiment of the present invention, the antibody is 3S9 or 4D12.

The invention also provides a method for manufacturing a medicament comprising admixing at least one of the monoclonal antibodies herein described, or a fragment or a homologue or a humanized or a chimeric version thereof with a medicament diluent or carrier. According to a specifically exemplified embodiment, the medicament comprises 3S9 or 4D12, or a fragment or a homologue thereof. According to a more specific embodiment, said medicament is used to prevent, alleviate or treat a disease associated with transplantation or autoimmune disease.

The invention, now being generally described, will be more readily understood by reference to the following examples, which are included merely for purposes of illustration of certain aspects and embodiments of the present invention and are not intended to limit the invention. All of the references mentioned herein are incorporated in their entirety by reference.

### EXAMPLES

### Example 1

### Materials

Animals

C57BL/6J (86) mice were originally purchased from Harlan Netherlands (Zeist, The Netherlands). (C57BL/6J 3 129/OIa) β₂-microglobulin-deficient (β²m⁻¹) mice were obtained from The Jackson Laboratory (Bar Harbor, Me.) or from Taconic Farms (Germany, N.Y.). Mice were bred in-house. To obtain dated pregnant mice, mice were mated for 15 h and the fetuses were removed from fetal day FD15 to FD18 (plug date=day 0). Fischer 344 rats were obtained from Iffa Credo (I'Arbresle, France). Mice and rats were treated and used in agreement with the institutional guidelines.

Media and Reagents

RPMI 1640 or DMEM media were supplemented with 10% FCS, 100 U/ml penicillin, 100 μg/ml streptomycin, 0.03% glutamine and 5×10⁻⁵ M β-mercaptoethanol (all from Life Technologies, Paisley, Scotland). These media will be further referred to as complete RPMI 1640 or complete DMEM medium, respectively. Purified human rIL-2 and rIL-7 were kindly provided by Dr. M. Gately (Hoffmann-La Roche, Nutley, N.J.) and Dr. S. Gillis (Immunex Corporation, Seattle, Wash.), respectively.

Antibodies

Monoclonal Abs used for staining were anti-CD3 (FITC-, APC-, and PerCP-conjugated, clone 145-2C11; PharMingen, San Diego, Calif.), anti-FcγRII/III (unconjugated, clone 2.4G2, rat IgG2b; provided by J. Unkeless, Mount Sinai School of Medicine, New York, N.Y.), anti-CD44 (PE-conjugated, clone IM7; PharMingen), anti-2B4 (unconjugated, clone 2B4; PharMingen), anti-NK1.1 (biotin- and PE-conjugated, clone PK136; PharMingen), anti-Ly49A (biotin-conjugated, clone A1; PharMingen), anti-Ly49C/I (biotin- and PE-conjugated, clone 5E6; PharMingen), and anti-Ly49C (biotin-conjugated, clone 4LO3311; provided by S. Lemieux, Institute Armand-Frappier, Quebec, Canada). The hybridoma cell line 4D11 secreting the anti-Ly49G2 was obtained from American Type Culture Col-lection (Manassas, Va.). The Ab was biotin-conjugated after purification by ammonium sulfate precipitation. FITC-conjugated polyclonal goat anti-rat 1g was obtained from PharMingen. Monoclonal Abs 4D12 and 3S9 were biotin- and FITC-conjugated after purification by adsorption column chromatography.

Preparation of Cell Suspensions

FD17 thymuses, FD17 spleens, and lymph nodes from 11-mo-old mice were removed and disrupted using a small Potter homogenizer. Spleens from 8- to 12-wk-old mice were removed and teased apart. Erythrocytes from spleens were lysed with 0.17 M NH₄Cl, and the remaining lymphocytes were washed three times with Dulbecco's PBS. Cells were counted with trypan blue to exclude dead cells. Thymocytes and splenocytes were suspended in RPMI 1640 medium supplemented with 10% FCS, 100 U/ml penicillin, 100 μg/ml streptomycin, 0.03% glutamine, and 5×10⁻⁵ M 2-ME (all obtained from Life Technologies, Paisley, U.K.). This medium will be further referred to as complete RPMI 1640 medium.

Skin was removed from killed adult mice and was freed of fat tissue followed by flotation, dermal side down, on 0.3% trypsin solution (Sigma, St. Louis, Mich.) at 4° C. for 18 h. Skin samples were pooled in complete DMEM medium containing 0.25% DNase (Boehringer Mannheim, Mannheim, Germany). Single cell suspensions were prepared by mechanical agitation and separated from dead cells by lympholyte-M (Cedarlane Laboratories Ltd., Hornby, Canada) density gradient centrifugation. Interface cells were collected and cultured overnight in complete RPMI 1640 medium supplemented with 250 U/ml IL-7 to allow recovery of surface expression of membrane proteins.

IL-2 stimulation

Purified human rIL-2 was provided by M. Gately (Hoffmann-LaRoche, Nutley, N.J.). Spleen and thymus cell suspensions were cultured in 24-well plates (Falcon; Becton Dickinson, Mountain View, Calif.) at 2 3 10 6 cells per well in 2 ml with complete RPMI 1640 medium at a final concentration of 1000 U/ml IL-2. After culture for 4 days in 5% CO₂ at 37° C., cells were harvested, washed twice, and counted with trypan blue.

Example 2

### Expression Ly49E and CD94JNKG2 on Murine NK Cells

The present example demonstrates the generation of antibodies to inhibitory receptors on natural killer cells and their characterization.

Methods

Flow Cytometric Analysis (FCA) and Sorting

Where indicated, freshly isolated adult splenocytes were depleted of B cells using sheep anti-mouse IgG Dynabeads (Dynal, Hamburg, Germany). To avoid aspecific binding, FcγR was blocked by preincubation of cells with saturating amounts of anti-FcγgRII/III mAb. Cells were incubated with the indicated mAbs for 45 min at 4° C. After washing, biotin-conjugated mAbs were revealed with second step streptavidin-APC (Becton Dickinson). To perform double-staining of NK cells, mAbs 5E6 and 4LO3311 were added 30 min before mAb 4D12. Cells were analyzed for fluorescence using a FACSCalibur flow cytometer (Becton Dickinson Immunocytometry Systems, Mountain View, Calif.) equipped with an argon laser (488 nm) and a helium-neon laser (540 nm) with the CellQuest software program (Becton Dickinson) for data acquisition and analysis. Propidium iodide was added to the cells (2 μg/ml) just before FCA. Gating was performed on propidium iodide-negative cells to exclude dead cells. Sorting was performed on a FACSVantage (Becton Dickinson) equipped with an argon laser.

Immunization and Screening

For the generation of mAb 4D12 (anti-Ly49E/C), 5-8×10⁵ IL-2-cultured FD17 thymocytes in PBS were injected twice i.p into Fischer rats with an interval of 3 wk. For the generation of mAb 3S9 (anti-NKG2A/C/E), 5-10×10⁶ IL-2-cultured splenocytes in PBS from adult mice were injected i.p. three times into a second group of Fischer rats. For both groups of rats, a final boost of 5-10×10⁵ cells was given i.v. 3 days before fusion of rat splenocytes and SP210 myeloma cells. Supernatants of growing hybridomas were screened as follows: human embryonic kidney T cells (HEK-T cells) transiently transfected with Ly49E cDNA in the expression vector pcDNA1.1 or HEK-T cells transiently cotransfected with the expression vectors BSRαEN encoding CD94 cDNA and NKG2A cDNA were incubated with the different supematants. The presence of Abs to these receptors was analyzed by flow cytometry through binding of FITC-conjugated anti-rat Ig polyclonal Ab. The 4D12 (anti-Ly49E/C) and 3S9 (anti-NKG2A/C/E) hybridomas were selected and cloned by limiting di-lution. Monoclonal Abs 4D12 and 3S9 are of the rat IgG2a(κ) and IgG2b(λ) isotype, respectively.

Immunoprecipitation, Deglycosylation, and Western Blotting

Where indicated, cells were surface biotinylated with 0.1 M D-biotin-N-hydroxysuccinimidester (Pierce, Rockford, Ill.) and lysed in 1% Nonidet

P-40 lysis buffer (1% Nonidet P-40, 1 mM EDTA, 50 mM Tris-HCl, 200 mM NaCl, 1% BSA, protease inhibitors). Nuclei were removed by centrifugation at 14,000 rpm at 4° C. for 30 min. Lysates were precleared with protein G-Sepharose 4 Fast Flow (Pharmacia Biotech, Uppsala, Sweden), incubated with specific mAb, and followed by incubation with protein G-Sepharose. Immunoprecipitates were washed with 0.5% Nonidet P-40 lysis buffer and separated on SDS-PAGE. For removal of N-linked sugars, immunoprecipitates were treated with N-glycosidase F using a deglycosylation kit according to the manufacturer's instructions (Boehringer Mannheim, Mannheim, Germany) and analyzed on SDS-PAGE. Blotting was performed on polyvinylidene difluoride membranes (Novex, San Diego, Calif.), and blots were blocked with 10% Western blocking reagent (Novex). Blots were incubated with streptavidin-conjugated HRP, and biotin-labeled proteins were visualized with precipitating HRP substrate (both obtained from Boehringer Mannheim).

Stimulation and Anti-Phosphotyrosine Detection

IL-2-cultured FD17 thymocytes (10-20×10⁶) containing ˜20% NK cells were harvested and resuspended at 6×10⁵ cells/ml in complete RPMI 1640 medium. Cells were stimulated with 0.01% H₂O₂ and 0.1 mM sodium orthovanadate (pervanadate) at 37° C for 20 min. Pervanadate is an inhibitor of protein tyrosine phosphatases, and treatment of cells with pervana-date induces protein tyrosine phosphorylation as described (48). Cells were lysed in 1% Nonidet P-40 lysis buffer containing 1 mM orthovanadate, and lysates were precipitated with mAb 4D12 (anti-Ly49E/C). Immunoprecipi-tates were separated on SOS-PAGE followed by Western blotting. Blots were incubated with 0.4 μg/ml HRP-conjugated anti-phosphotyrosine mAb (clone PY99) (Santa Cruz Biotechnology, Santa Cruz, Calif.) and revealed with precipitating HRP substrate.

RT-PCR

Trizol (Life Technologies) was added to sorted cells, and RNA was extracted according to the manufacturer's instructions. Before reverse transcription, digestion of DNA was performed with deoxyribonuclease I (Life Technologies). cDNA was synthesized with oligo(dT) as primer using the Superscript kit (Life Technologies). For HPRT, a housekeeping enzyme, oligonucleotides were GTA ATG ATC AGT CAA CGG GGG AC (SEQ ID NO:1: sense primer) and CCA GCA AGC TTG CAA CCT TAA CCA (SEQ ID NO:2: antisense primer). For CD94, oligonucleotides were GTG CAA TTG TTA CTT TAT TTC C (SEQ ID NO:3: sense primer) and CTG AGA ATT CTG GAA ATA AAT C (SEQ ID NO:4: antisense primer). For NKG2A, primers were GGT TGA CTC GAG CCA TGA GTA ATG AAC GCG TCA C (SEQ ID NO:5: sense primer) and CGT GAA TCT AGA TTA TCA GAT GGG GAA TTT ACA CT (SEQ ID NO:6: antisense primer). PCR amplification was performed using a 96-well thermocycler (Omnigene, Hy-baid Teddington, U.K.) with 35 cycles of 94° C. for 30 s, 55° C. for 30 s, and 72*C for 1 min (HPRT and CD94), with 35 cycles of 94° C. for 30 s, 57° C for 30 s, and 72° C. for 1 min (NKG2A). In each PCR, water and 50 ng mouse genomic DNA were included as negative controls.

cDNA Constructs and Transfection of HEK-T Cells

We received plasmids encoding Ly-49A, Ly-49B, Ly-49C, Ly-49D, and Ly49H from F. Takel (Terry Fox Laboratory, Vancouver, Canada) (11, 12, 49) and plasmids encoding Ly-49E, Ly-49F, and Ly-49G1 from W. M. Yokoyama (Mount Sinai Medical Center, New York, N.Y.) (13). The constructs for eukaryotic expression of Ly49 were prepared by subcloning the Ly49 cDNAs into the pcDNA1.1 expression vector (Invitrogen BV, Leek, The Netherlands). In addition, the Ly49E cDNA was subcloned into the BSRαEN expression vector (provided by J. C. Ryan, University of California, San Francisco, Calif.). The sense primers for cloning Ly49E in pcDNA1.1 and BSRαEN contained the ATG start codon present at position 99 of the Ly49E cDNA (13). The Ly491 cDNA in the eukaryotic expres-sion vector pTS was provided by M. Bennett (University of Texas South-western Medical Center, Dallas, Tex.) (50). For cloning CD94 and NKG2A into the BSRαEN expression vector, a PCR was performed on cDNA gen-erated from FD17 thymocytes. For CD94, oligonucleotides were GGT TGA CTC GAG ATA CCA TGG CAG TTT CTA GGA TCA CTC GG (SEQ ID NO:7: sense primer) and CGT GM TCT AGA GAA ACA TTT MA TAG GCA GTT TC (SEQ ID NO:8: antisense primer). For NKG2A, primers were GGT TGA CTC GAG CCA TGA GTA ATG AAC GCG TCA C (SEQ ID NO:9: sense primer) and CGT GAA TCT AGA TTA TCA GAT GGG GAA TTT ACA CT (SEQ ID NO:10: anti-sense primer). pME18S plasmids encoding NKG2C, NKG2E-hemaglutinin (HA), and DAP12 cDNAs were provided by D. Raulet (University of Cal-ifornia, Berkeley, Calif.). HEK-T cells were transiently transfected using the calcium phosphate-mediated transfection (51). HEK-T cells transfected with the empty ex-pression vector or with the human IL-2Ra cDNA encoded in the pcDNA1.1 expression vector were used as a negative control. After 2 days, HEK-T cells were harvested and analyzed by flow cytometry or used for immunoprecipitation.

Cell Mediated Cytotoxicity

Tumor targets used were the NK-sensitive cell line, RMA (H-2^{b}; obtained from A. Kruisbeek, Amsterdam, The Netherlands) and the TAP-2 mutant derivative of RMA, RMA-S (provided by A. Geldhof, Vrije Universiteit Brussel, Brussels, Belgium). Target cells (1×10⁶) were labeled with 100 μCi ⁵¹Cr (Amersham International, Buckinghamshire, U.K.) for 60 min at 37° C. Cells were washed three times. Effector cells were incubated with 5 μg/ml mAb 2B4 at 4° C. As shown previously, the cytotoxicity of fetal NK cells can be triggered by preincubation of NK cells with mAb 2B4 (41, 44, 45). After 1 h, the unbound mAb was removed by washing the cells. Graded effector cell numbers were added in duplicate to 10³ tumor cells in V-bottom wells of a 96-well plate in a final volume of 100 μl/well. After incubation for 4 h at 37° C, 75 μl of supernatant was removed from each well. Optiphase Supermix (225 μl; Wallac, Turku, Finland) was added to the supernatants, and radioactivity was measured using a 96-well scintillation counter (Microbeta; Wallac). The spontaneous release of radioactiv-ity was determined in wells without effector cells, and the maximal release in wells in which target cells were lysed by addition of 1% Triton X-100 at the start of incubation. Percent specific lysis was calculated as 100×(experimental−spontaneous release)/(maximal−spontaneous release).

Results

Generation of a mAb Recognizing Ly49E/C and a mAb Recognizing NKG2A/C/E

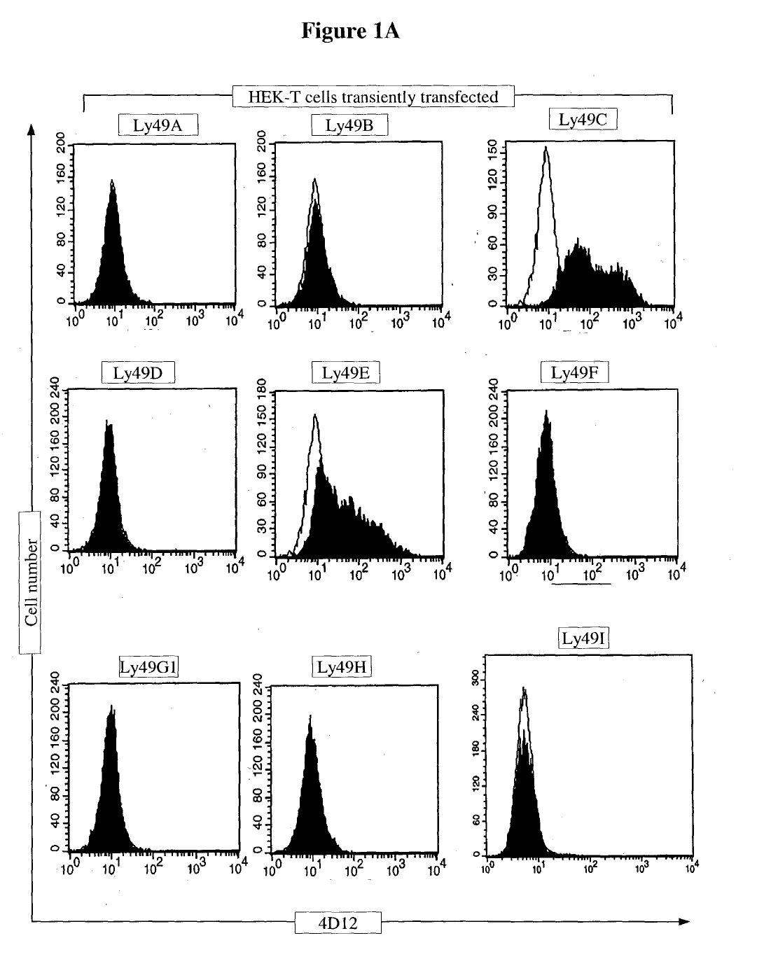

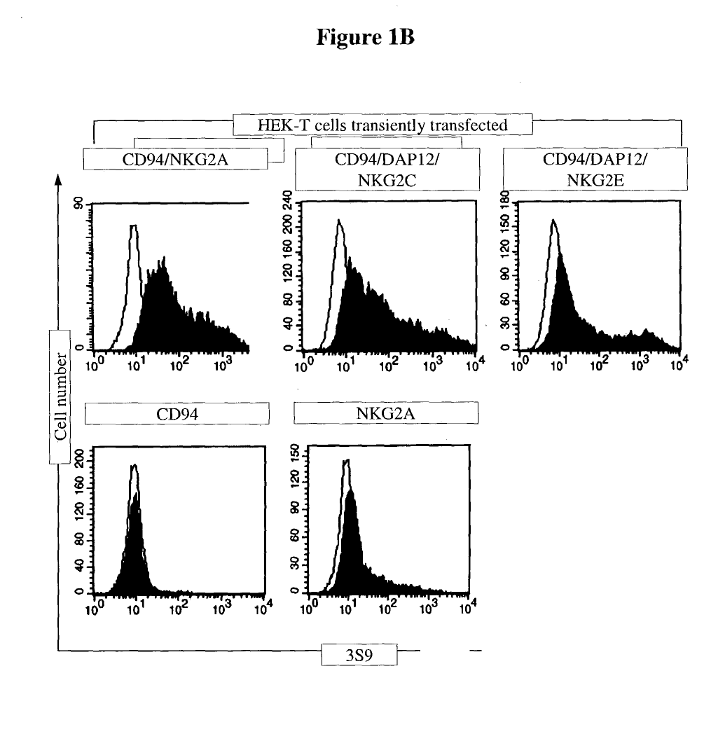

To produce an anti-Ly49E mAb, Fischer rats were immunized with IL-2-cultured murine FD17 thymocytes. One hybridoma, secreting mAb 4D12, was selected by flow cytometry for its binding to Ly49E-transfected cells. The specificity of mAb 4D12 was analyzed on HEK-T cells transiently transfected with plasmids encoding different Ly49 cDNAs. As shown in FIG. 1A, mAb 4D12 binds to Ly49E and Ly49C but not to Ly49A, B, D, F, G1, H, or I. As expected, mAbs Al (anti-Ly49A), 12A8 (anti-Ly49D), 4D11 (anti-Ly49G), and 5E6 (anti-Ly49C/I) stained HEK-T cells transfected with Ly49A, D, G1, and I, respectively. Expression of Ly49F and Ly49H was shown by using a rat antiserum generated against B6 adult splenocytes. We could not demonstrate expression of Ly49B using the rat antiserum (data not shown).

We also generated a mAb against NKG2 using IL-2-cultured adult splenocytes as an immunogen. The mAb 389 specifically recognizes HEK-T cells cotransfected with NKG2A and CD94 cDNAs as well as HEK-T cells transfected with NKG2A cDNA alone. It did not bind HEK-T cells transfected with CD94 cDNA alone (FIG. 1B). We observed low expression of NKG2A alone on the surface of transfected HEK-T cells (FIG. 1B). The extracellular domains of NKG2C and NKG2E are >90% identical with the carbohydrate recognition domain of NKG2A (9). Therefore, the specificity of mAb 3S9 to NKG2C and NKG2E was also tested (FIG. 1B). Although it has not been shown that murine NKG2C and NKG2E associate with DAP12, cotransfection of DAP12 with CD94/NKG2C and CD94/NKG2E enhances receptor expression (9). FIG. 1B shows that mAb 3S9 binds to HEK-T cells transfected with CD94/DAP12/NKG2C and CD941DAP12/NKG2E.

Biochemical Analysis of Ly49E and NKG2A

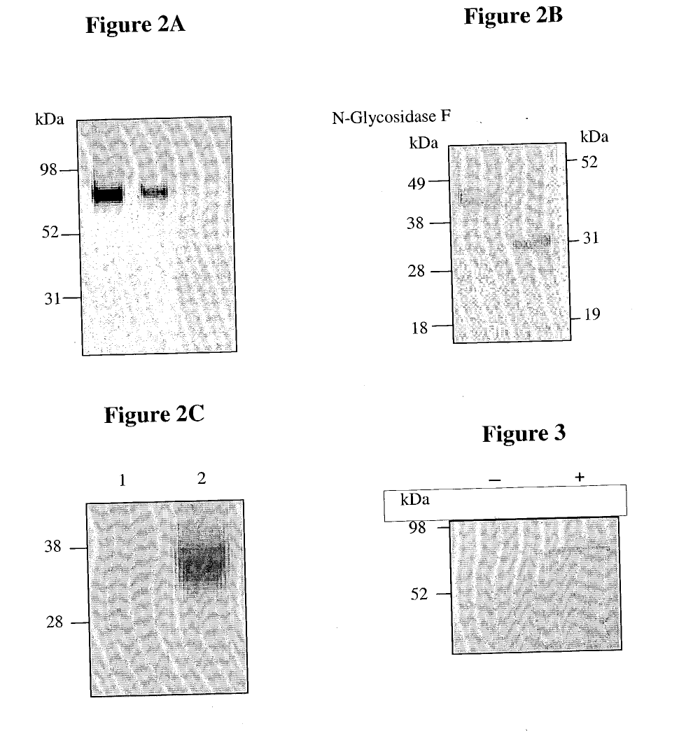

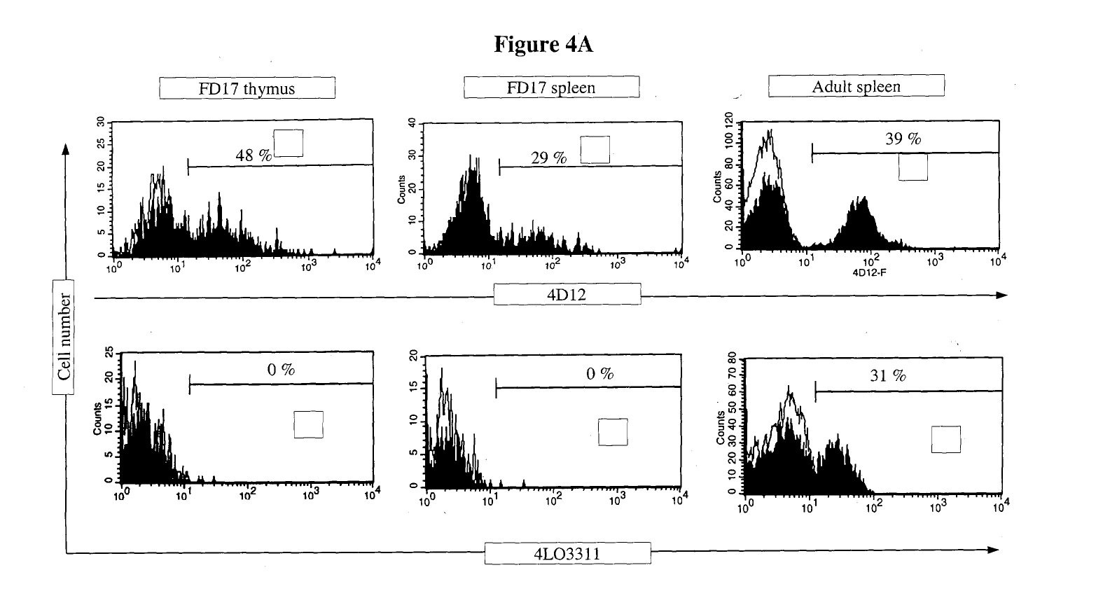

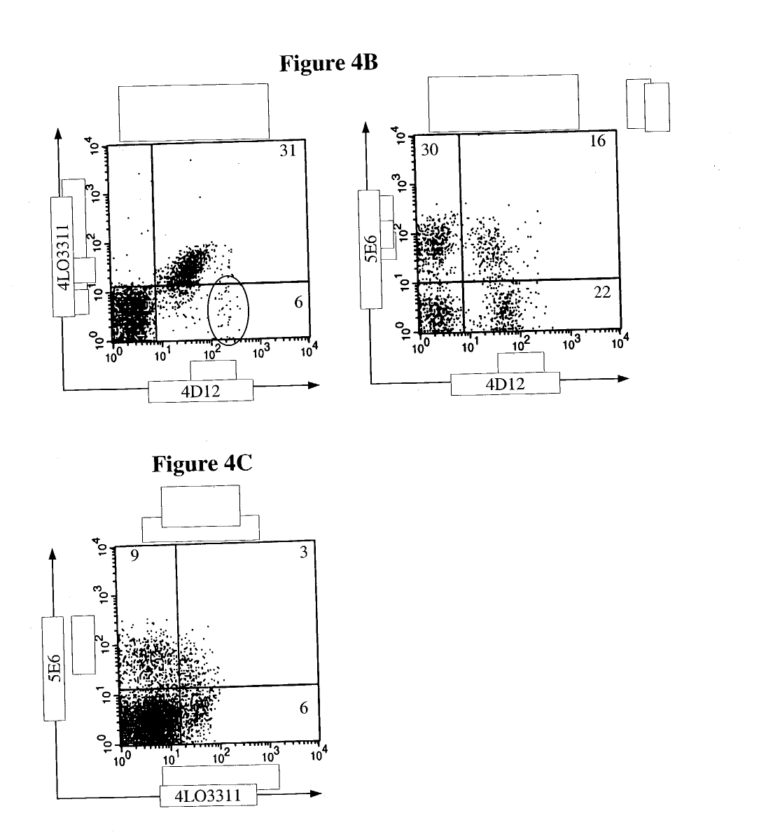

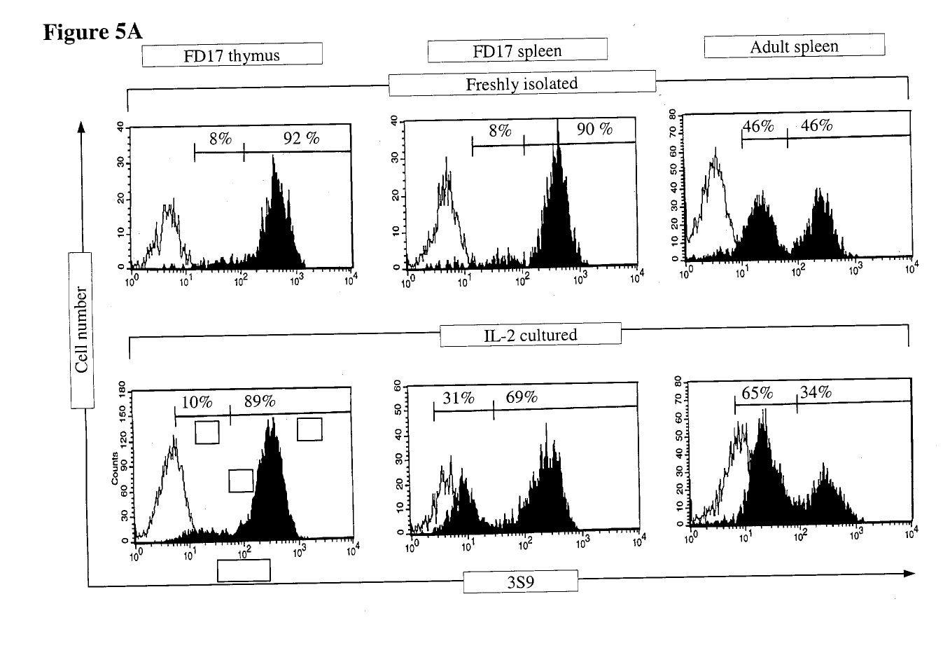

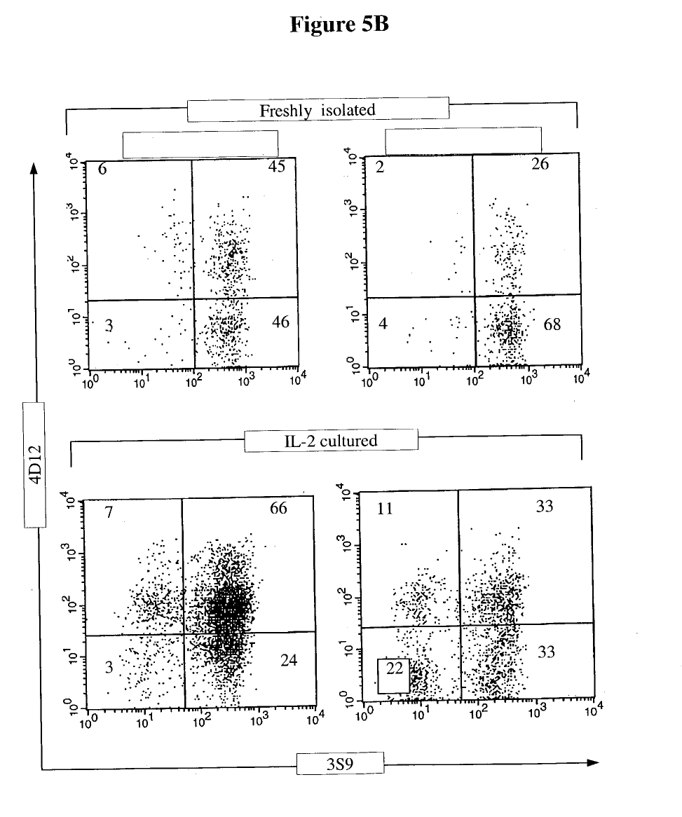

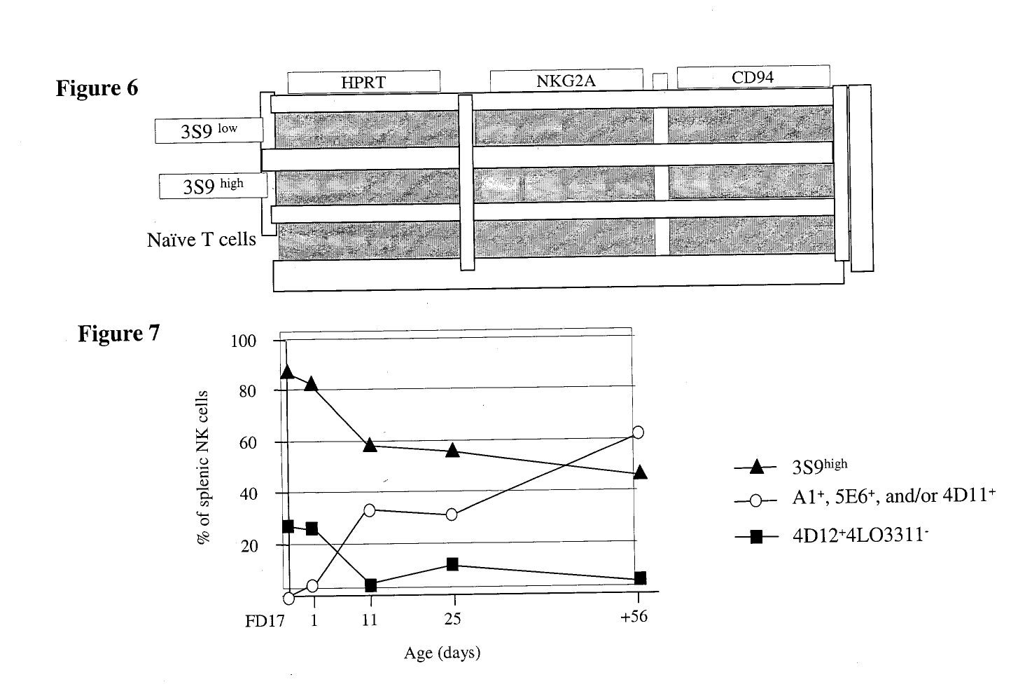

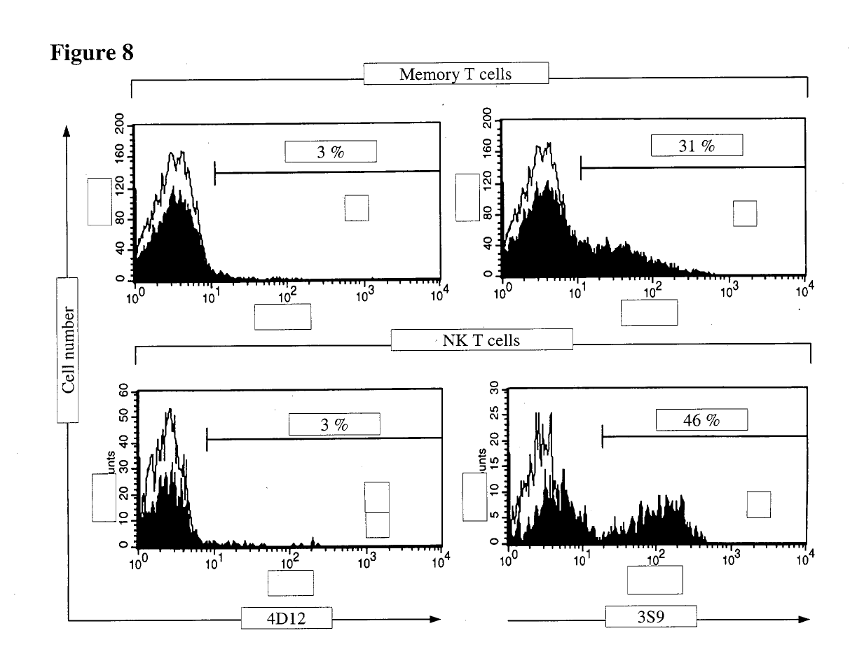

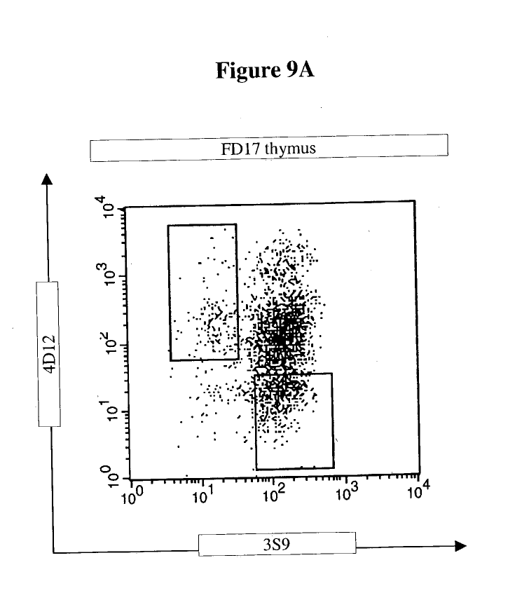

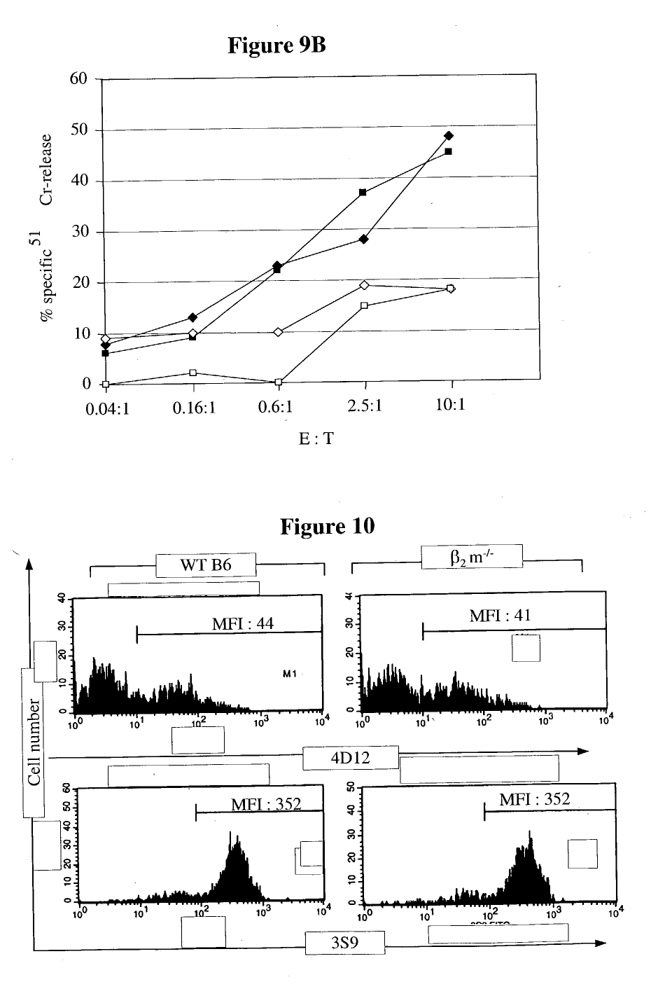

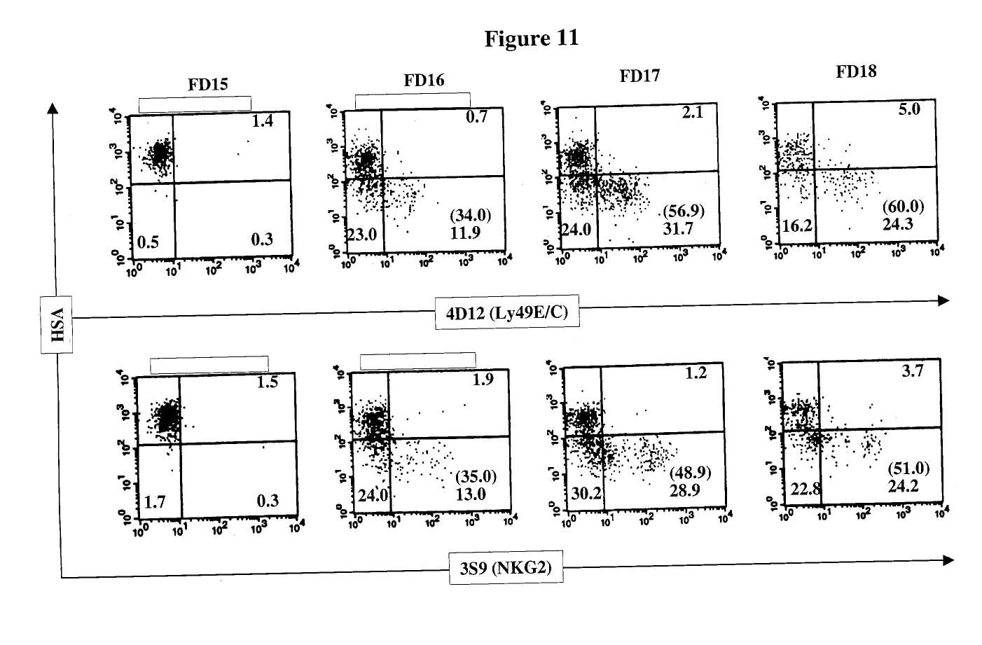

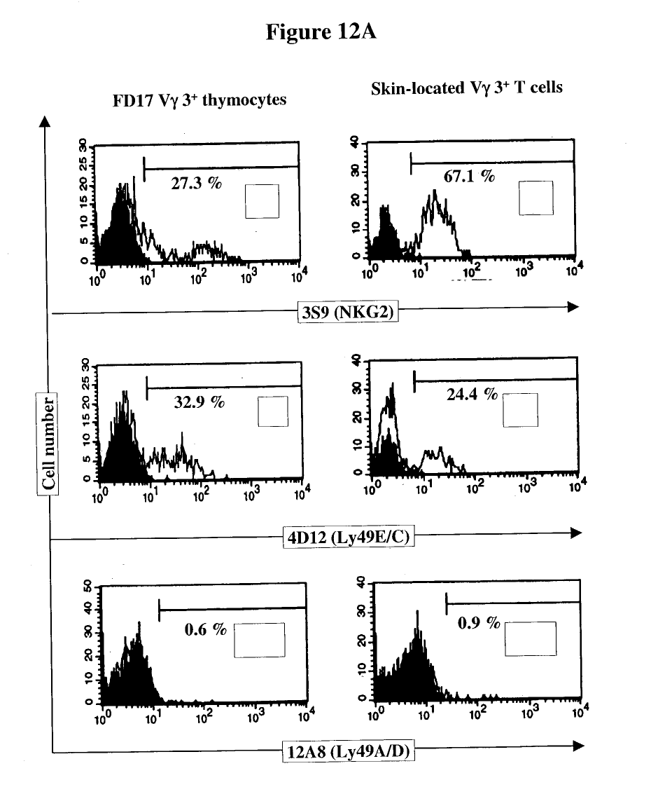

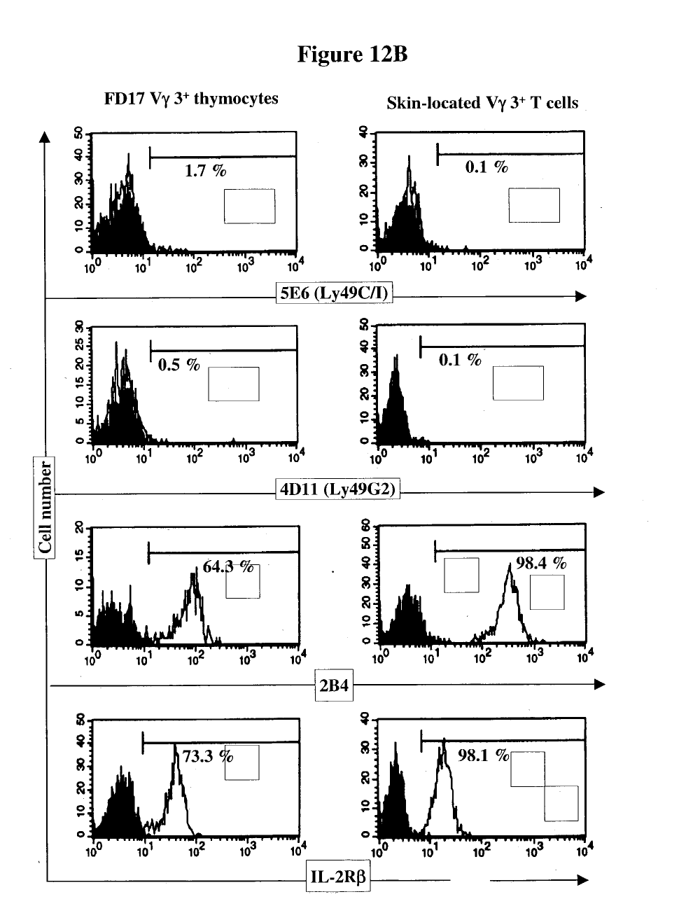

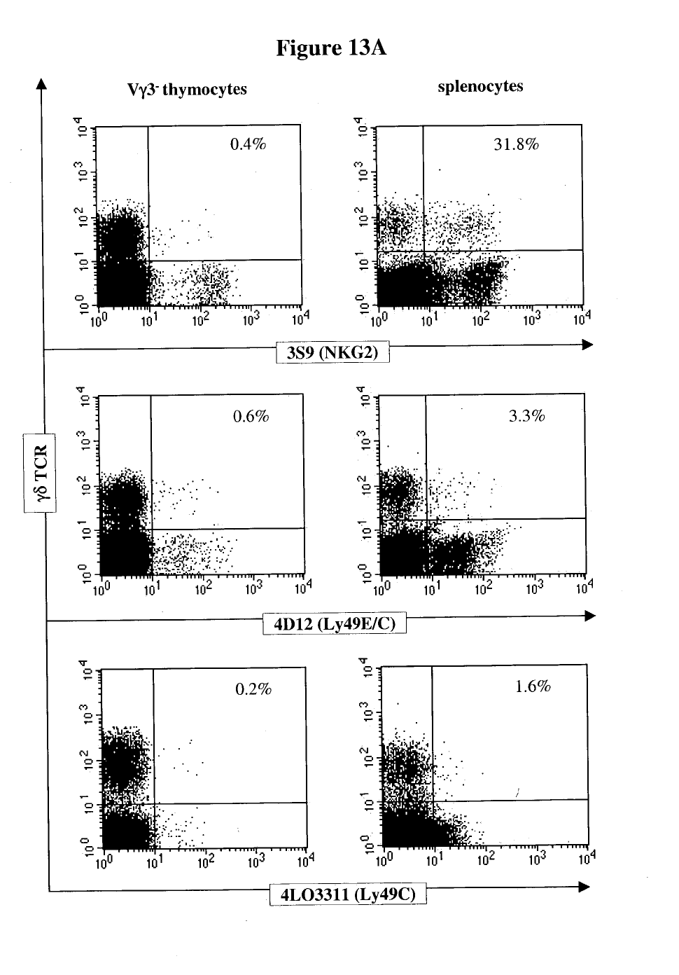

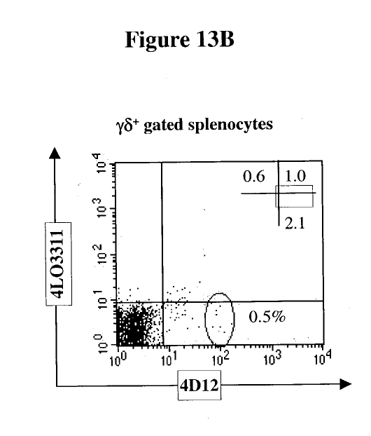

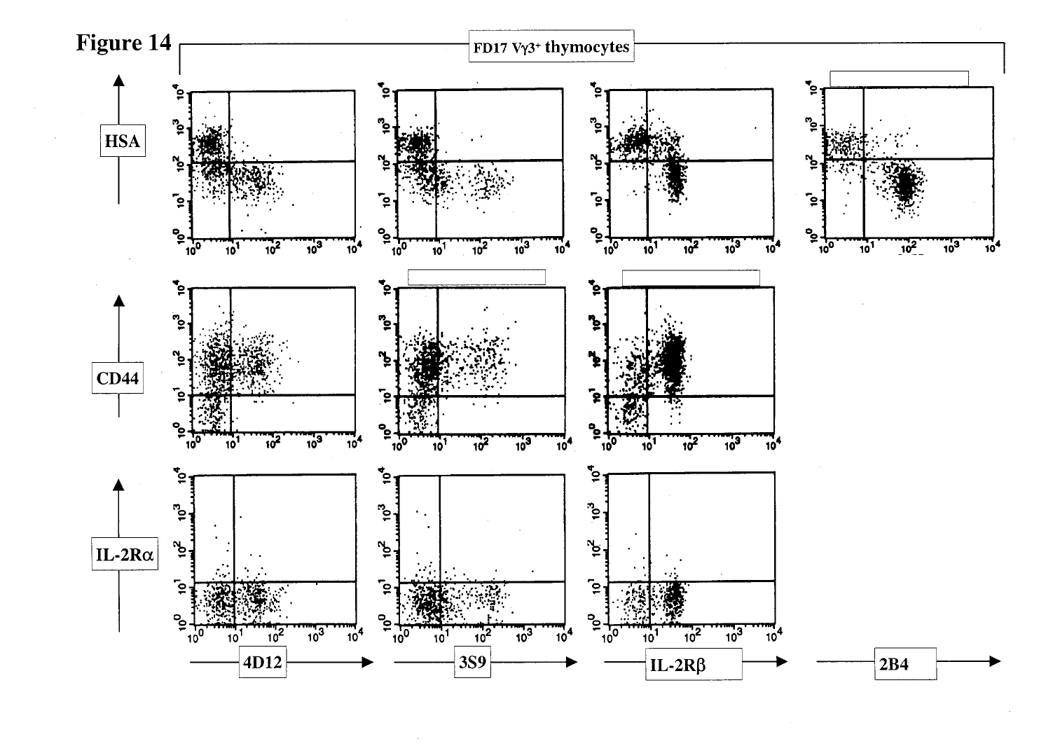

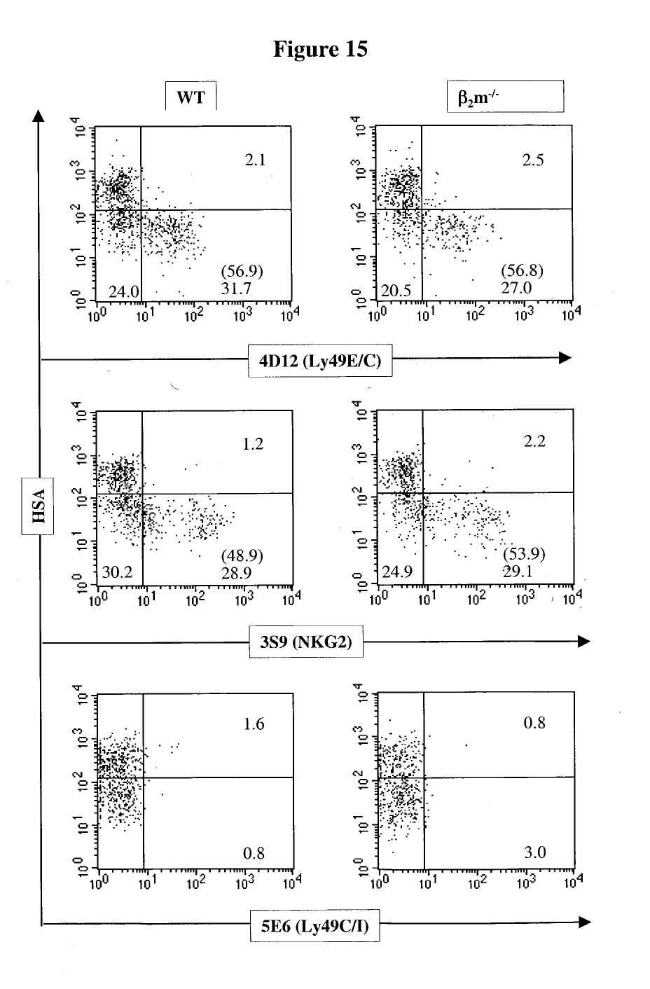

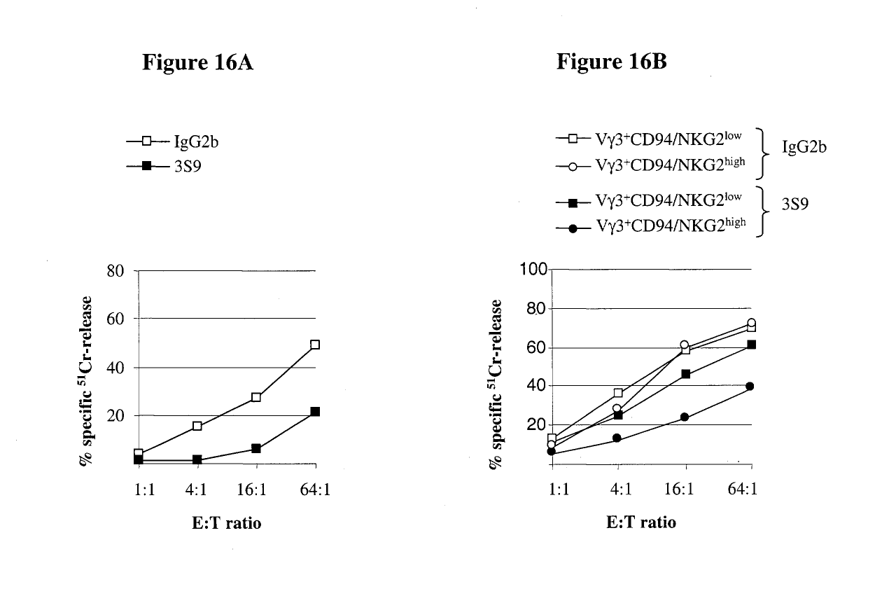

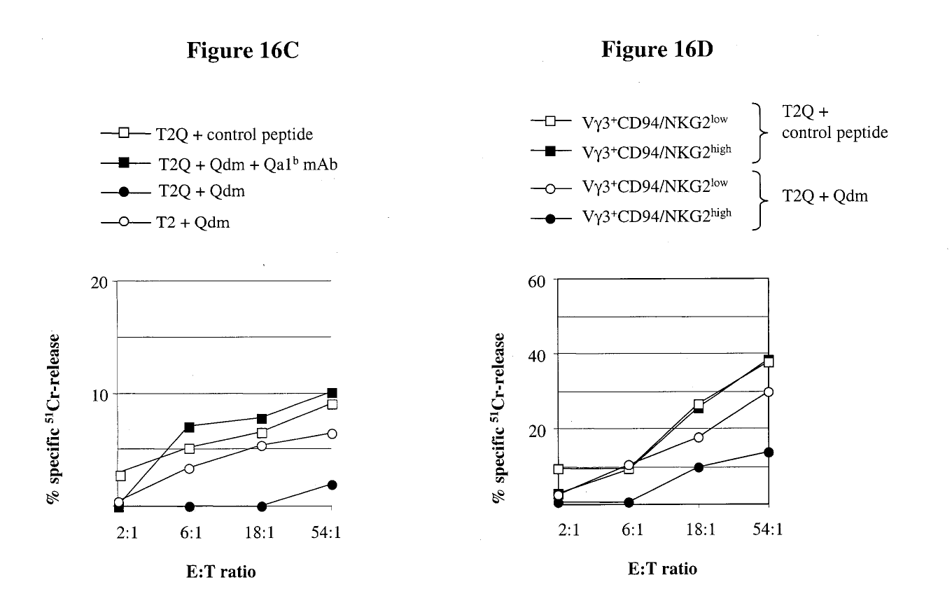

To analyze the biochemical characteristics of Ly49E, the protein was immunoprecipitated with mAb 4D12 both from lysates of biotin-labeled HEK-T cells transfected with the plasmid BSRαEN encoding Ly49E cDNA and from lysates of IL2-cultured FD17 thymocytes. SDS-PAGE under nonreducing conditions showed that mAb 4D12 immunoprecipitates a ˜90-kDa protein from FD17 thymocytes and Ly49E-transfected HEK-T cells. HEK-T cells transfected with the empty vector were used as a negative control (FIG. 2A). Immunoprecipitation of lysates from Ly49E transiently transfected HEK-T cells with mAb 4D12 followed by SDS-PAGE under reducing conditions identified a protein with a molecular mass of ˜46 kDa. Removal of N-linked sugars revealed a protein backbone of ˜31 kDa (FIG. 2B), which is in agreement with the predicted molecular mass of the monomer (13). These data show that Ly49E is expressed as a homodimer with; 46-kDa subunits, each containing ˜15-kDa N-linked carbohydrates.

Lysates from HEK-T cells expressing CD94/NKG2A were immunoprecipitated with mAb 3S9. SDS-PAGE under reducing conditions and Western blot analysis showed that NKG2A migrates as a ˜38-kDa protein (FIG. 2C). The extracellular domain of NKG2A contains five potential N-linked glycosylation sites (8). This property could explain the difference between the observed molecular mass and the predicted molecular mass (27 kDa) (8).

Stimulation of Ly49E Results in Tyrosine Phosphorylation

The presence of one ITIM consensus sequence within the cytoplasmic domain of Ly49E suggests an inhibitory role for this receptor. To investigate whether the tyrosine residue within the ITIM could be phosphorylated, IL-2-cultured FD17 thymocytes were stimulated with pervanadate or were left untreated. Cells were lysed and immunoprecipitated with mAb 4D12. The immunoprecipitates were separated by SDS-PAGE under nonreducing conditions, and blots were incubated with anti-phosphotyrosine mAb.

FIG. 3 shows a protein band at ˜90 kDa, demonstrating that Ly49E can be phosphorylated upon pervanadate stimulation.

Ly49E is Expressed on a Subpopulation of Fetal and Adult NK Cells.

The above data showed that mAb 4D12 recognizes the Ly49E receptor, with cross-reactivity for Ly49C. To outline further the phenotype of fetal and adult NK cells, we analyzed the expression of Ly49E by FCA. Therefore, freshly isolated FD17 splenocytes, FD17 thymocytes, and adult splenocytes were analyzed for their ability to bind mAb 4D12 by gating on CD3⁻ NK1.1⁺ cells.

Monoclonal Ab 4D12 recognized 50±4%, 30±2%, and 36±4% (average % 6 SD from more than three independent experiments) of NK cells from FD17 thymocytes, FD17 splenocytes, and adult splenocytes, respectively (FIG. 4A). Analysis of 4-day IL-2-cultured FD17 thymocytes, splenocytes, and adult splenocytes revealed that 68±6%, 50±5%, and 41±4% (average % 6 SD from more than three independent experiments), respectively, of CD3⁻ NK1.1⁺ cells were stained by mAb 4D12 (data not shown).

FIG. 4A also shows that mAb 4LO3311, specifically recognizing Ly49C (35), does not stain fetal NK cells. Therefore, the present data demonstrate that Ly49E is expressed on a considerable sub-population of fetal NK cells. Colabeling of adult NK cells with mAb 4D12 (anti-Ly49E/C) and mAb 4LO3311 (anti-Ly49C) revealed that 6% of freshly isolated adult NK cells were single positive for 4D12 (FIG. 4B, lower right quadrant of the left dot plot).

However, as the staining intensity of mAb 4LO3311 on B6 NK cells is low (FIG. 4B and S. Lemieux, unpublished observation) it is reasonable to assume that only the bright 4D12 single positive NK cells, of which the frequency is 1% (FIG. 4B, oval area in the left dot plot), are really negative for 4LO3311 and resemble Ly49C⁻ E⁺ NK cells. As mAbs 4LO3311 (anti-Ly49C) and 5E6 (ant-Ly49C/I) have overlapping binding specificity for Ly49C, we expected a similar percentage of 4D12 single positive cells after colabeling of adult NK cells with mAb 4D12 plus mAb 5E6. However, in this case we observed 22% of 4012 single positive NK cells (FIG. 4B). As mAb 4LO3311 does not recognize Ly49E (Ref. 35 and data not shown), a possible explanation for this discrepancy could be that mAbs 4LO3311 and 4D12 also recognize an un-known Ly49 molecule, which is not or only weakly recognized by mAb 5E6. Costaining of B cell-depleted splenocytes with mAbs 4LO3311 and 5E6 revealed a 4LO3311 single positive population when gated on CD3⁻ cells (FIG. 4C). FCA showed that 28% of CD3⁻ cells were NK1.11⁺ (data not shown).

Expression of CD94/NKG2A/C/E on Fetal and Adult NK Cells

FCA using mAb 3S9 was performed on freshly isolated thymocytes and splenocytes from FD17 mice and on adult splenocytes. FIG. 5A shows that ˜90% of uncultured fetal thymic as well as splenic NK cells expressed high levels of CD94/NKG2, whereas >50% of NK cells from adult spleen were CD94/NKG2^{high}. The remaining fetal and adult NK cells were not completely negative for 3S9 binding and showed low fluorescence intensity. FIG. 6A further reveals that after 4 days of IL-2 culture, the percentage of the CD94/NKG2^{low} population increased in both fetal and adult splenic CD32NK1.1⁺ cells as compared with uncultured NK cells.

FIG. 5B shows that binding of mAbs 4D12 and 3S9 on freshly isolated and IL2-cultured fetal CD32NK1.11 cells revealed four distinct subpopulations.

To obtain additional evidence that the low staining with mAb 3S9 (anti-NKG2) of a subpopulation of NK cells is not due to aspecific binding, but correlates with low expression of the CD94/NKG2 receptor, semiquantitative RT-PCR for NKG2A and CD94 was performed on sorted 3S9^{low} and 3S^{high} subpopulations of uncultured splenic NK cells. As a negative control, semiquantitative RT-PCR for NKG2A and CD94 was performed on naive T cells (CD3⁻CD44⁻3S9⁻) sorted from lymph nodes (FIG. 6). The results show that both NKG2A and CD94 transcripts were clearly present in 3S9^{low} cells, but at lower levels as compared with 3S9 cells.

Expression of Ly49E and CD94/NKG2 on Developing NK Cells

Expression of Ly49E during NK cell ontogeny was analyzed by determining the percentage of 4D12⁻ cells by gating on 4LO3311-negative NK cells. FIG. 7 shows that 27-29% of splenic NK cells from FD17 and 1 day postnatal mice were 4D12 single positive. From the second week alter birth, the percentage of 4D12 single positive cells decreased to 5-10%. As discussed previously, if only the bright 4D12 single positive NK cells, which are negative for 4LO3311, resemble Ly49C⁻ E⁺ NK cells, the percentage of Ly49E⁺ NK cells decreased to 1-3% from the second week after birth (data not shown). In contrast, the frequency of NK cells positive for Ly49A, C/I, and/or G2 increased after birth. Consistent with a previous study (30), the frequency of NK cells expressing high levels of CD94/NKG2 decreased from ˜90% in FD17 mice to ˜50% in adult mice (FIG. 7). Also the median fluorescence intensity of the CD94/NKG2^{high} subpopulation decreased during NK cell development (data not shown).

Expression of Ly49E and CD94/NKG2 on Memory T Cells and NK T Cells

A significant population of memory CD81T lymphocytes express the inhibitory receptors Ly49A, C/I, F, and G2, and this percentage increases with age (52). In 11 mo-old mice 12% of memory T cells (phenotypically defined as CD3⁺ CD44^{high}) were positive for staining with mAb 4D12 (data not shown), but only 3% of memory T cells were 4D12⁺ 4LO3311⁻ (FIG. 8), showing that the putative inhibitory receptor Ly49E is expressed on a minor population of peripheral memory T cells. Interestingly, ˜30% of memory T lymphocytes expressed CD94/NKG2 as shown by binding of mAb 3S9 (FIG. 8). We also examined NK T cells. These cells have been shown to coexpress CD3 and NK1.1, to be CD4⁺ or CD4⁻ CD8⁻, and to express inhibitory Ly49 receptors (53-56). Approximately 10% of NK T cells (phenotypically defined as CD3^{int} NK1.1⁺) stained with mAb 4D12 (data not shown), but only 3% were 4D12⁺4LO3311⁻ (FIG. 8). So, in contrast to Ly49A and Ly49G2, which are expressed on, respectively, 11 and 17% of NK T cells (data not shown), Ly49E is expressed on a very small population of NK T cells. Staining with mAb 3S9 showed that ˜50% of NK T cells express high levels of CD94/NKG2. The remaining NK T cells have low expression levels of CD94/NKG2 (FIG. 8).

Lysis of RMA-S target cells by fetal NK cells expressing either Ly49E or CD94/NKG2

In search of a functional role of the Ly49E receptor on fetal NK cells, we first sorted Ly49E⁻ and Ly49E⁺ subpopulations from IL-2-cultured FD17 thymocytes and assessed their lytic capacity against RMA and the TAP-deficient RMA-S target cells. The Ly49E⁻ and Ly49E⁺ subpopulations were equally capable to differentially lyse RMA-S cells (data not shown). This might be explained by the fact that a major part of the cells of both the Ly49E⁻ and Ly49E⁺ subpopulations express CD94/NKG2 (FIG. 5B). Therefore, we sorted Ly49E⁺ CD94/NKG2 low NK cells from 4-day IL-2-cultured FD17 thymocytes. As a control, also Ly49E⁻ CD94/NKG2^{high} NK cells were sorted (FIG. 9A). Sorted cells were cultured in the presence of IL-2 for an additional period of 3 days to remove the mAbs from the cell surface. RMA and RMA-S target cells were still differentially lysed by the sorted Ly49E⁺ CD94/NKG2^{low} cells, and the differential killing was comparable to that of sorted Ly49E⁻ CD94/NKG2^{high} cells (FIG. 9B). This differential lysing of RMA and RMA-S cells by the Ly49E⁺CD94/NKG2^{low} cells could be due either to the expression of the Ly49E receptor or to the low expression of CD94/NKG2. To further assess whether Ly49E is involved in the resistance of RMA target cells, blocking studies by incubating effector cells with F(ab′)2 of anti-Ly49E mAb (mAb 4D12) were performed. This did not reverse the resistance of RMA target cells to lysis (data not shown). It is possible that mAb 4D12 is not able to functionally block the interaction of Ly49E with its ligand or, alternatively, that RMA cells do not express the ligand for the Ly49E receptor.

Expression Levels of Ly49E and CD94/NKG2 on NK Cells from WT and β₂ m⁻¹ Mice

The ligand for Ly49E is still unknown. Because the expression level of several other Ly49 receptors on NK cells is down-regulated in vivo by the presence of the corresponding MHC class I ligands (37, 38, 57-61), we investigated whether H-2^{b} MHC class I molecules are able to influence the expression levels of Ly49E and CD94/NKG2A receptors. Because Ly49E is expressed as the only member of the Ly49 family on fetal NK cells, the expression level of Ly49E was compared between fetal thymic NK cells from WT B6 mice and β₂ m⁻¹ mice. FIG. 10 shows that the expression level of Ly49E was not down-regulated on NK cells from WT B6 mice as compared with 2 m⁻1 mice, suggesting that the ligand for Ly49E is not expressed in WT B6 mice. Vance et al. demonstrated that CD94/NKG2 heterodimers recognize the nonclassical MHC Qa-1^{b} molecules (9, 27). Because the expression of Qa-1 b is β₂m-dependent (62), this would suggest a higher expression level of CD94/NKG2 in β₂ m ⁻1 mice com-pared with WT. Interestingly, we found that the relative fluorescence intensity of CD94/NKG2 was the same in both strains of mice (FIG. 10). Also the percentages of CD94/NKG2^{low} and CD94/NKG2^{high} cells were not significantly different (data not shown).

### Example 3

### Expression Ly49E and CD94/NKG2 on Fetal Thymic and Adult Epidermal TCR Vγ3 Lymphocytes

The present example demonstrates that the antibodies of Example 2 also detect Ly49E and CD94/NKG2 receptors on TCR Vγ3 lymphocytes.

Methods

Flow Cytometric Analysis (FCA) and Sorting.

Where indicated, freshly isolated thymocytes and adult splenocytes were depleted of CD4 T cells using unconjugated L3T4 mAb and sheep anti-mouse IgG Dynabeads (Dynal, Hamburg, Germany). To avoid aspecific binding, FcγR was blocked by preincubation of cells with saturating amounts of anti-FcγRII/III mAb. Cells were incubated with the indicated mAbs for 45 min at 4° C. After washing, biotin-conjugated mAbs were revealed with second step streptavidin-APC or streptavidin-PE (Becton Dickinson). Cells were analyzed for fluorescence using a FACSCalibur flow cytometer (Becton Dickinson Immunocytometry Systems, Mountain View, Calif.) equipped with an argon laser (488 nm) and a helium-neon laser (633 nm) with the CellQuest software program (Becton Dickinson) for data acquisition and analysis. Propidium iodide was added to the cells (2 μg/ml) just before FCA. Gating was done on propidium iodide negative cells to exclude dead cells. Sorting was performed on a FACS Vantage (Becton Dickinson) equipped with an argon laser.

Cell Mediated Cytotoxicity.

Tumor targets used were the mastocytoma P815 (H-2K^{d}, D^{d}) and TAP-deficient T2Q cells transfected with the Qa1^{b} gene and untransfected T2 cells (68) (Dr. C. Brooks, The Medical School, Newcastle, United Kingdom). 1×10⁶ target cells were labeled with 100 μCi ⁵¹Cr (Amersham International, Buckinghamshire, England) in serum-free medium for 60 min at 37° C. Cells were washed three times. As shown previously, the cytotoxicity of FD17 thymocytes can be triggered by pre-incubation with mAb 2B4 (45). Cytolytic activity against P815 was tested in the presence of 3S9 mAb or isotype control antibody both at a final concentration of 30 μg/ml When untransfected T2 cells or Qa1^{b}-transfected T2Q cells were used, these cells were incubated at 26° C. with 30 μM Qdm peptide (SEQ ID No: 11: AMAPRTLLL) (Dr. C. Brooks, The Medical School, Newcastle, United Kingdom, (47)) or with a an unrelated peptide (SEQ ID No: 12: ESIINFEK) or 18 h before the killing assay. 30 μM of peptide was also added during ⁵¹Cr-labeling and during the cytotoxicity assay. In blocking studies, T2Q target cells loaded with Qdm peptide were pre-incubated with anti-Qa1^{b} mAb at room temperature for 1 h and assayed for lysis by Vg3 T cells in the presence of 20 μg/ml anti-Qa1b mAb. Graded effector cell numbers were added in duplicate to 10³ tumor cells in V-bottomed wells of a 96 well plate in a final volume of 100 μl/well. After incubation for 4 h at 37° C., 75 μl supernatant was removed from each well. 225 μl Optiphase Supermix (Wallac, Turku, Finland) was added to the supernatants and radioactivity was measured using a 96-well scintillation counter (Microbeta, Wallac). The spontaneous release of radioactivity was determined in wells without effector cells, and the maximal release in wells in which target cells were lysed by addition of 1% Triton X-100 at the start of incubation. Percent specific lysis was calculated as: 100×(experimental−spontaneous release)/(maximal−spontaneous release).

Results

Ly49E and NKG2 Receptors are Expressed by TCR Vγ3 Cells During Differentiation in the Fetal Thymus.

Earlier analysis by our group has shown that during fetal life immature TCR Vγ3^{low}HSA^{high} cells differentiate in the thymus into mature TCR Vγ3 cells with a Vγ3^{high} HSA^{low} phenotype (69). Recently, we have generated two mAbs against NK receptors: mAb 4D12 recognizing Ly49E with cross-reactivity to Ly49C, and mAb 3S9 against NKG2A/C/E (70). To investigate if Ly49E/C and NKG2 NK receptors are expressed on TCR Vγ3 thymocytes, thymocytes from FD15 to FD18 were freshly isolated and analyzed by gating on Vγ3⁺ T cells using flow cytometry. FIG. 11 shows that FD15 Vγ3 thymocytes are immature (HSA^{high}) and did not express Ly49E/C or NKG2 receptors. From FD16, part of the TCR Vγ3 thymocytes become mature (HSA^{low}) and Ly49E/C and NKG2 receptors were expressed on a subpopulation of these mature cells. The percentages of mature TCR Vγ3 thymocytes expressing Ly49E/C or NKG2 increased from approximately 35% at FD16 to approximately 60% for Ly49E/C and approximately 50% for NKG2 at FD17 to FD18.

The above data show that both Ly49E/C and NKG2 receptors are expressed on a subpopulation of TCR Vγ3 thymocytes. A more detailed analysis of the NK receptor expression on thymic TCR Vγ3⁺ lymphocytes is shown in FIG. 12. Since the highest percentage of mature TCR Vγ3 thymocytes is found at FD17 (data not shown), we focused on this stage of development. As fetal Vγ3 thymocytes are the precursors of Vγ3⁺T cells in the epidermis of adult mice (71), we also analyzed the expression of NK receptors by skin-located Vγ3 T cells in parallel. The NK receptor expression was analyzed by gating on Vγ3⁺ cells using flow cytometry. Whereas NKG2 was detected on approximately 30% of total FD17 TCR Vγ3⁺ thymocytes, NKG2⁺ cells comprised approximately 70% of epidermal Vγ3⁺ T cells. mAb 4D12 (anti-Ly49E/C) stained one third of TCR Vγ3⁺ T cells isolated from FD17 thymus or adult skin. Analysis of both thymic and epidermal Vγ3 T cells revealed that mAb 5E6, recognizing Ly49C and 1, did not stain Vγ3 T cells. Therefore, the present data demonstrate that fetal thymic and skin-located Vγ3 T cells expressed Ly49E but not Ly49C. We also failed to detect expression of Ly49A, D, or G2 on Vγ3 T lymphocytes. To exclude the possibility that a small subpopulation of TCR Vγ3 thymocytes expressed Ly49 receptors, FD17 thymocytes were cultured with r-IL-2 for four days. This resulted in an increase in the percentage of Vγ3 T cells (72). However, no expression of other Ly49 molecules could be observed (data not shown). Further phenotypic analysis revealed that 2B4 and IL-2Rβ were expressed on the majority of thymic TCR Vγ3⁺ lymphocytes and on all skin-located Vγ3 T cells (FIG. 12). Neither thymic nor epidermal Vγ3 T cells expressed the NK receptor DX5 (data not shown). Approximately 10% of FD17 TCR Vγ3⁺ thymocytes expressed the NK receptor NK1.1 and this percentage increased to approximately 40% after IL-2 culture (data not shown).

Expression of CD94/NKG2 and Ly49E on γδ T Cells Other than Vγ3 T Cells.

The expression of CD94/NKG2 and Ly49E was also analyzed on neonatal (day 0) thymic and on adult splenic γδ T cells. As a low percentage of Vγ3 cells is still present in neonatal thymus, Vγ3⁺ cells were gated out in these samples. CD94/NKG2 was detected on approximately 30% of splenic γδ T cells and on only 0.4% of neonatal thymic Vγ3 γδ⁺ T cells (FIG. 13A). The low frequency of CD94/NKG2⁺ neonatal thymic γδ T cells could not be due to their immaturity, since 40% of neonatal Vγ3γδ⁺ T cells expressed low levels of HSA, representing mature γδ T cells (data not shown). FIG. 13A shows that approximately 3% of adult splenic γδ T cells and <1% of neonatal Vγ3γδ⁺ T cells expressed Ly49E/C. As mAb 4D12 recognizes both Ly49E and Ly49C, we performed staining with mAb 4LO3311 alone and double-staining with mAbs 4D12 and 4LO3311. Staining with mAb 4LO3311 alone revealed that approximately 0.2% and 1.6% of respectively neonatal thymic and adult splenic γδ T cells expressed Ly49C. By gating on TCR γδ⁺ splenocytes, co-staining demonstrated that 2.1% of these γδ T cells were 4D12 single-positive. As previously reported (70), it is reasonable to assume that only the bright 4D12 single-positive γδ T cells, which represent 0.5% of splenic γδ T cells (FIG. 13B, oval area in dot plot), resemble Ly49C E⁺ γδ T cells. As we are not able to distinguish more than 3 different fluorochromes, we could not combine 4D12 and 4LO3311 mAbs on fetal Vγ3γδ⁺ thymocytes. In conclusion, these data demonstrate that Ly49E and CD94/NKG2 are less frequently expressed on neonatal thymic and adult splenic γδ T cells compared to Vγ3 T cells.

Vγ3⁺ Thymocytes Expressing NK Receptors Exhibit a Memory Phenotype.

Because it appeared that Ly49E and NKG2A/C/E NK receptors are exclusively expressed on mature TCR Vγ3 thymocytes, we examined if expression of 2B4 and IL-2Rβ by Vγ3 thymocytes also correlated with a mature phenotype. As shown in FIG. 14, both 2B4 and IL-2Rβ receptors were mainly expressed on mature HSA^{low} TCR Vγ3 thymocytes. Furthermore, we questioned whether expression of NK receptors on mature TCR Vγ3 thymocytes parallels with a memory phenotype by these cells. FIG. 14 shows that all Ly49E⁺, NKG2⁺ or IL-2Rβ⁺ Vγ3⁻ T cells expressed CD44, which is indicative of a memory phenotype. In accordance with this, essential all Ly49E⁺, NKG2⁺, or IL-2Rβ⁺ Vγ3⁺ T cells did not express CD25.

Ly49E and NKG2A/C/E Expression by TCR Vγ3⁺ Lymphocytes is Independent on MHC Class I Molecules.

Coles et al. demonstrated that expression of Ly49 receptors on memory CD8⁻ TCR αβ cells is predominantly dependent upon MHC class I expression (52). To analyze if expression of Ly49E and CD94/NKG2 receptors on Vγ3 T cells is also affected by loss of MHC class I molecules, we compared the expression of these receptors on TCR Vγ3 thymocytes isolated from FD17 wild type versus β₂ m⁺ 6 mice. As illustrated in FIG. 15, we observed similar percentages of Ly49E and NKG2 positive cells, and also similar expression levels of these receptors on mature FD17 Vγ3 thymocytes from β₂⁺ mice compared to wild type mice. This shows that Ly49E and NKG2 expression on Vγ3 cells is not dependent upon MHC class I expression. As it has been demonstrated that the expression of Ly49C on NK cells is upregulated in β₂M⁻ mice compared to WT mice (58, 47), we wanted to exclude the possibility that the similar percentages of 4D12⁺Vγ3 T cells in both mice were due to an increased expression of Ly49C in β₂m⁺ mice. For this purpose, the expression of Ly49C on Vγ3 T cells was compared between WT and β₂M⁺ mice. FIG. 15 demonstrates that Ly49C is not expressed on FD17 Vγ3 lymocytes from both WT and β₂m⁺ mice. Therefore, these data show that similar frequencies of Ly49E⁺ Vγ3 T cells are present in WT and γ₂ mice.

CD94/NKG2-Mediated Inhibition of Tumor Cell Lysis by TCR Vγ3 T Cells.

To examine if the CD94/NKG2 receptors expressed on Vγ3 T cells are functional, we analyzed their role in the cytotoxic activity of these cells. TCR Vγ3⁺ cells were sorted from FD17 thymocytes after 3 days of IL-2 culture. After an additional culture with IL-2 for 3 days, to remove mAbs from the cell surface, Vγ3 T lymphocytes were used as effector cells in different cytotoxic assays. As illustrated in FIG. 16A, FcγR⁺ P815 target cells were at least four times less susceptible to lysis after pre-treatment of Vγ3⁺ T cells with mAb 3S9 compared to isotype control mAb, indicating that cross-linking of CD94/NKG2 results in inhibition of the cytotoxicity of Vγ3 cells. Next, we analyzed the cytotoxic activity of CD94/NKG2^{low} and CD94/NKG2^{high} sorted subsets of Vγ3⁺ T cells. Part (approximately 20%) of sorted CD94/NKG2^{low} Vγ3 cells upregulated CD94/NKG2 expression during the additional IL-2-culture, while expression of CD94/NKG2 on sorted CD94/NKG2 Vγ3 T cells was stable (data not shown). As demonstrated in FIG. 16B, P815 targets were less susceptible to lysis by Vγ3⁺CD94/NKG2high T cells as compared to Vγ3⁺CD94/NKG2^{low} T cells after pre-treatment of these effector cells with anti-NKG2 mAb. To obtain more evidence that CD94/NKG2 functions as an inhibitory receptor on Vγ3 T cells, we performed a cytotoxic assay using target cells which are able to present the ligand of CD94/NKG2 (47, 73). The ligand for CD94/NKG2 is the Qdm-peptide presented in the context of Qa1^{b} molecule. Qdm is derived from the leader sequence of classical MHC class I molecules and forms a complex with the non-classical MHC Qa1^{b} molecule peptide in a TAP-dependent manner (9, 27, 28, 74, 75). T20 cells, transfected with Qa1 ^{b}, were pre-incubated in the presence of Qdm peptide or unrelated peptide and were used as target cells. As illustrated in FIG. 16C, the lysis of T20 target cells by Vγ3 T cells was clearly inhibited by addition of Qdm peptide but not unrelated peptide. The inhibition of Vγ3 killing activity was not due to non-specific effects of Qdm peptide alone, since untransfected T2 targets incubated with Qdm peptide were efficiently lysed by Vγ3 T cells. Addition of anti-Qa1b mAb to T20 targets loaded with Qdm reversed inhibition of cytolytic activity of Vγ3 T cells (FIG. 160). FIG. 16D shows that T2Q target cells, pre-incubated with Qdm, were less susceptible to lysis by the CD94/NKG2^{high} subset of Vγ3 T cell compared to the CD94/NKG2^{low} subset of Vγ3 T cells. The slight reduction in the killing of T20 targets incubated with Qdm by CD94/NKG2^{low} Vγ3 cells as compared to the killing of T20 targets incubated with control peptide, is probably due to low expression of CD94/NKG2 on the majority of these effector cells, and to the upregulation of CD94/NKG2 on part of these cells. T20 targets incubated with unrelated peptide were equally killed by both effector cells. In conclusion, these data demonstrate the significance of Qa1^{b}/Qdm recognition by CD94/NKG2, and show that CD94/NKG2 expressed on TCR Vγ3 T cells functions as an inhibitory receptor following recognition of Qdm presented by Qa1^{b}.

### REFERENCES

1. Kärre, K., H. Ljunggren, G. Piontek, and R. Kiessling. 1986. Selective rejection of H-2-deficient lymphoma variants suggests alternative immune defence strategy. Nature 319:675.

2. Ljunggren, H. G., and K. Kärre. 1990. In search of the ‘missing self’: MHC molecules and NK recognition. Immunol. Today 11:237.

3. Bennett, M. 1987. Biology and genetics of hybrid resistance. Adv. Immunol. 41:333.

4. Murphy, W. J., V. Kumar, and M. Bennett. 1987. Acute rejection of murine bone marrow allografts by natural killer cells and T cells. Differences in kinetics and target antigens recognized. J. Exp. Med. 166:1499.

5. Lanier, L. L. 1998. NK cell receptors. Annu. Rev. Immunol. 16:359.

6. Vance, R. E., D. M. Tanamachi, T. Hanke, and D. H. Raulet. 1997. Cloning of a mouse homolog of CD94 extends the family of C-type lectins on murine natural killer cells. Eur. J. Immunol. 27:3236.

7. Ho, E. L., J. W. Heusel, M. G. Brown, K. Matsumoto, A. A. Scalzo, and W. M. Yokoyama. 1998. Murine Nkg2d and Cd94 are clustered within the natural killer complex and are expressed independently in natural killer cells. Proc. Nat. Acad. Sci. U.S.A. 95:6320.

8. Lohwasser, S., P. Hande, D. L Mager, and F. Takei. 1999. Cloning of murine NKG2A, B and C: second family of C-type lectin receptors on murine NK cells. Eur. J. Immunol. 29:755.

9. Vance, R. E., A. M. Jamieson, and D. H. Raulet. 1999. Recognition of the class 1b molecule Qa-1 (b) by putative activating receptors CD94/NKG2C and CD94/NKG2E on mouse natural killer cells. J. Exp. Med. 190:1801.

10. Yokoyama, W. M., P. J. Kehn, D. I. Cohen, and E. M. Shevach. 1990. Chromosomal location of the Ly-49 (A1, YE1/48) multigene family: genetic association with the NK 1.1 antigen. J. Immunol. 145:2353.

11. Wong, S., J. D. Freeman, C. Kelleher, D. Mager, and F. Takei. 1991. Ly-49 multigene family. New members of a superfamily of type II membrane proteins with lectin-like domains. J Immunol. 147:1417.

12. Brennan, J., D. Mager, W. Jefferies, and F. Takei. 1994. Expression of different members of the Ly-49 gene family defines distinct natural killer cell subsets and cell adhesion properties. J. Exp. Med. 180:287.

13. Smith, H. R., F. M. Karlhofer, and W. M. Yokoyama. 1994. Ly-49 multigene family expressed by IL-2-activated NK cells. J. Immunol. 153:1068.

14. McQueen, K. L., J. D. Freeman, F. Takei, and D. L. Mager. 1998. Localization of five new Ly49 genes, including three closely related to Ly49c. Immunogenetics 48:174.

15. Burshtyn, D. N., A. M. Scharenberg, N. Wagtmann, S. Rajagopalan, K. Berrada, T. Yi, J. P. Kinet, and E. 0. Long. 1996. Recruitment of tyrosine phosphatase HCP by the killer cell inhibitor receptor. Immunity 4:77.

16. Nakamura, M. C., E. C. Niemi, M. J. Fisher, L. D. Shultz, W. E. Seaman, and J. C. Ryan. 1997. Mouse Ly-49A interrupts early signaling events in natural killer cell cytotoxicity and functionally associates with the SHP-1 tyrosine phosphatase. J. Exp. Med. 185:673.

17. Mason, L. H., P. Gosselin, S. K. Anderson, W. E. Fogler, J. R. Ortaido, and D. W. McVicar. 1997. Differential tyrosine phosphorylation of inhibitory versus activating Ly-49 receptor proteins and their recruitment of SHP-1 phosphatase. J. Immunol. 159:4187.

18. Houchins, J. P., L. L. Lanier, E. C. Niemi, J. H. Phillips, and J. C. Ryan. 1997. Natural killer cell cytolytic activity is inhibited by NKG2-A and activated by NKG2-C. J. Immunol. 158:3603.

19. Lanier, L. L., B. C. Corliss, J. Wu, C. Leong, and J. H. Phillips. 1998. Immunoreceptor DAP12 bearing a tyrosine-based activation motif is involved in activating NK cells. Nature 391:703.

20. Lanier, L. L., B. Corliss, J. Wu, and J. H. Phillips. 1998. Association of DAP12 with activating CD94/NKG2C NK cell receptors. Immunity 8:693.

21. Smith, K. M., J. Wu, A. B. Bakker, J. H. Phillips, and L. L. Lanier. 1998. Ly-49D and Ly-49H associate with mouse DAP12 and form activating receptors. J. Immunol. 161:7.

22. Wu, J., Y. Song, A. B. Bakker, S. Bauer, T. Spies, L. L. Lanier, and J. H. Phillips. 1999. An activating immunoreceptor complex formed by NKG2D and DAP10. Science 285:730.

23. Gosselin, P., L. H. Mason, J. Willette Brown, J. R. Ortaldo, D. W. MoVicar, and S. K. Anderson. 1999. Induction of DAP12 phosphorylation, calcium mobilization, and cytokine secretion by Ly49H. J. Leukoc. Biol. 66:165.

24. Braud, V. M., D. S. J. Allan, C. A. O'Cailaghan, K. S6derstrom, A. D'Andrea, G. S. Ogg, S. Lazetic, N. T. Young, J. I. Bell, J. H. Phillips, L. L. Lanier, and A. J. McMichael. 1998. HLA-E binds to natural killer cell receptors CD94/NKG2A, B and C. Nature 391:795.

25. Borrego, F., M. Ulbrecht, E. H. Weiss, J. E. Coligan, and A. G. Brooks. 1998. Recognition of human histocompatibility leukocyte antigen (HLA)-E complexed with HLA class I signal sequence-derived peptides by CD94/NKG2 confers protection from natural killer cell-mediated lysis. J. Exp. Med. 187:813.

26. Lee, N., M. Llano, M. Carretero, A. Ishitani, F. Navarro, M. Lopez Botet, and D. E. Geraghty. 1998. HLA-E is a major ligand for the natural killer inhibitory receptor CD94/NKG2. Proc. Natl. Acad. Sci. U.S.A. 95:5199.

27. Vance, R. E., J. R. Kraft, J. D. Altman, P. E. Jensen, and D. H. Raulet. 1998. Mouse CD94/NKG2A is a natural killer cell receptor for the nonclassical major histocompatibility complex (MHC) class I molecule Qa-1 (b). J. Exp. Med. 188:1841.

28. Colterill, L. A., H. J. Stauss, M. M. Millrain, D. J. C. Pappin, D. Rahman, B. Canas, P. Chandler, A. Stackpoole, E. Simpson, P. J. Robinson, and P. J. Dyson. 1997. Qa-1 Interaction and T cell recognition of the Qa-1 determinant modifier peptide. Eur. J. Immunol. 27:2123.

29. Salcedo, M., P. Bousso, H. G. Ljunggren, P. Kourilsky, and J. P. Abastado. 1998. The Qa-1b molecule binds to a large subpopulation of murine NK cells. Eur. J. Immunol. 28:4356.

30. Salcedo, M., F. Colucci, P. J. Dyson, L. A. Cotterill, F. A. Lemonnier, P. Kourilsky, J. P. Di Santo, H. G. Ljunggren, and J. P. Abastado. 2000. Role of Qa1b-binding receptors in the specificity of developing NK cells. Eur. J. Immunol. 30:1094.

31. Hanke, T., H. Takizawa, C. W. McMahon, D. H. Busch, E. G. Pamer, J. D. Miller, J. D. Altman, Y. Liu, D. Cado, F. A. Lemonnier, P. J. Bjorkman, and D. H. Raulet. 1999. Direct assessment of MHC class I binding by seven Ly49 inhibitory NK cell receptors. Immunity 11:67.

32. Michaelsson, J., A Achour, M. Salcedo, A. Kase Sjostrom, J. Sundback, R. A. Harris, and K. Karre. 2000. Visualization of inhibitory Ly49 receptor specificity with soluble major histocompatibility complex class I tetramers. Eur. J. Immunol. 30:300.

33. Liu, J., M. A. Morris, P. Nguyen, T. C. George, E. Koulich, W. C. Lai, J. D. Schatzle, V. Kumar, and M. Bennett. 2000. Ly49l NK cell receptor transgene inhibition of rejection of H2b mouse bone marrow transplants. J. immunol. 164:1793.

34. Mason, L. H., J. R. Ortaldo, H. A. Young, V. Kumar, M. Bennett, and S. K. Anderson. 1995. Cloning and functional characteristics of murine large granular lymphocyte-1: a member of the Ly-49 gene family (Ly-49G2). J. Exp. Med. 182:293.

35. Brennan, J., S. Lemieux, J. D. Freeman, D. L. Mager, and F. Takei. 1996. Heterogeneity among Ly-49C natural killer (NK) cells: characterization of highly related receptors with differing functions and expression patterns. J. Exp. Med. 184:2085.

36. Raulet, D. H., W. Held, I. Correa, J. R. Dorfman, M. F. Wu, and L. Corral. 1997. Specificity, tolerance and developmental regulation of natural killer cells defined by expression of class I-specific Ly49 receptors. Immunol. Rev. 155:41.

37. Salcedo, M., A. D. Diehl, M. Y. Olsson Alheim, J. Sundback, L. Van Kaer, K. Karre, and H. G. Ljunggren. 1997. Altered expression of Ly49 inhibitory receptors on natural killer cells from MHC class I-deficient mice. J. Immunol. 158:3174.

38. Karlhofer, F. M., R. Hunziker, A. Reichlin, D. H. Margulies, and W. M. Yokoyama. 1994. Host MHC class I molecules modulate in vivo expression of a NK cell receptor. J. Immunol. 153:2407.

39. Olsson, M. Y., K. Karre, and C. L. Sentman. 1995. Altered phenotype and function of natural killer cells expressing the major histocompatibility complex receptor Ly-49 in mice transgenic for its ligand. Proc. Natl. Acad. Sci. USA. 92:1649.

40. Dorfman, J. R., and D. H. Raulet. 1996. Major histocompatibility complex genes determine natural killer cell tolerance. Eur. J. Immunol. 26:151.

41. Sivakumar, P. V., M. Bennett, and V. Kumar. 1997. Fetal and neonatal NK1.1(+) Ly-49(−) cells can distinguish between major histocompatibility complex class I-hi and class I-lo target cells: evidence for a Ly-49-independent negative signaling receptor. Eur. J. Immunol. 27:3100.

42. Manday and Sykes, Curr. Opin. ImmunoL 10:532-538 (1998)).

43. Lanier, Immunity 6:371-78 (1997).

44. Toomey, J. A., S. Shrestha, S. A. de la Rue, F. Gays, J. H. Robinson, Z. M. A. Chrzanowska-Lightowlers, and C. G. Brooks. 1998. MHC class I expression protects target cells from lysis by Ly49-deficient fetal NK cells. Eur. J. Immunol. 28:47.

45. Van Beneden, K., A. De Creus, V. Debacker, J. De Boever, J. Plum, and G. Leclercq. 1999. Murine fetal natural killer cells are functionally and structurally distinct from adult natural killer cells. J. Leukoc. Biol. 66:625.

46. Yokoyama and Seaman, Ann Rev Immun 11:613-35 (1993).

47. Toomey, J. A., M. Salcedo, L. A. Cotterill, M. M. Millrain, Z. Chrzanowska Lightowlers, J. Lawry, K. Fraser, F. Gays, J. H. Robinson, S. Shrestha, P. J. Dyson, and C. G. Brooks. 1999. Stochastic acquisition of Qa1 receptors during the development of fetal NK cells in vitro accounts in part but not in whole for the ability of these cells to distinguish between class I-sufficient and class I-deficient targets. J. Immunol. 163:3176.

48. O'Shea, J. J., D. W. McVicar, T. L. Bailey, C. Burns, and M. J. Smyth. 1992. Activation of human peripheral blood T lymphocytes by pharmacological induction of protein-tyrosine phosphorylation. Proc. Natl. Acad. Sci. USA. 89:10306.

49. Brennan, J., G. Mahon, D. L. Mager, W. A. Jefferies, and F. Takei. 1996. Recognition of class I major histocompatibility complex molecules by Ly49: specificities and domain interactions. J. Exp. Med. 183:1553.

50. Stoneman, E. R., M. Bennett, J. An, K. A. Chesnut, E. K. Wakeland, and J. B. Scheerer. 1995. Cloning and characterization of 5E6 (Ly-49C), a receptor molecule expressed on a subset of murine natural killer cells. J. Exp. Med. 182:305.

51. Chen, C., and H. Okayama. 1987. High-efficiency transformation of mammalian cells by plasmid DNA. Mol. Cell. Biol. 7:2745.

52. Coles, M. C., C. W. McMahon, H. Takizawa, and D. H. Raulet. 2000. Memory CD8 T lymphocytes express inhibitory MHC-specific Ly49 receptors. Eur. J. Immunol. 30:236.

53. Bendelac, A., M. N. Rivera, S.-H. Park, and J. H. Roark. 1997. Mouse CD1-specific NK1 T cells: development, specificty, and function. Annu. Rev. ImmunoL 15:535.

54. Roland, J., and P.-A. Cazenave. 1992. Ly-49 antigen defines an ab TCR population in i-IEL with an extrathymic maturation. Int. Immunol. 4:699.

55. MacDonald, H. R., R. K. Lees, and W. Held. 1998. Developmentally regulated extinction of Ly-49 receptor expression permits maturation and selection of NK1.1⁺T cells. J. Exp. Med. 187:2109.

56. Ortaldo, J. R., R. Winkler Pickett, A. T. Mason, and L. H. Mason. 1998. The Ly-49 family: regulation of cytotoxicity and cytokine production in murine CD3⁺cells. J. Immunol. 160:1158.

57. Held, W., J. R. Dorfman, M. F. Wu, and D. H. Raulet. 1996. Major histocompatibility complex class I-dependent skewing of the natural killer cell Ly49 receptor reportoire. Eur. J. Immunol. 26:2286.

58. Dorfman, J. R., J. Zerrahn, M. C. Coles, and D. H. Raulet. 1997. The basis for self-tolerance of natural killer cells in beta2-microglobulin- and TAP-1-mice. J. Immunol 159:5219.

59. Hoglund, P., J. Sundback, M. Y. Olsson Alheim, M. Johansson, M. Saicedo, C. Ohlen, H. G. Ljunggren, C. L. Sentman, and K. Karre. 1997. Host MHC class I gene control of NK-cell specificity in the mouse. Immunol. Rev. 155:11.

60. Andersson, M., S. Freland, M. H. Johansson, R. Wallin, J. K. Sandberg, B. J. Chambers, B. Christensson, U. Lendahl, S. Lemieux, M. Salcedo, and H. G. Ljunggren. 1998. MHC class I mosaic mice reveal insights into control of Ly49C inhibitory receptor expression in NK cells. J. Immunol. 161:6475

61. Salcedo, M., M. Andersson, S. Lemieux, L. Van Kaer, B. J. Chambers, and H. G. Ljunggren. 1998. Fine tuning of natural killer cell specificity and maintenance of self tolerance in MHC class I-deficient mice. Eur. J. Immunol. 28:1315.

62. Robinson, P. J., P. J. Travers, A. Stackpoole, L. Flaherty, and H. Djaballah. 1998. Maturation of Qa-1b class I molecules requires beta 2-microglobulin but is TAP independent. J. Immunol. 160:3217.

63. Mullinax et al., BioTechniques 12(6):864-869(1992)

64. Held, W., D. Cado, and D. H. Raulet. 1996. Transgenic expression of the Ly49A natural killer cell receptor confers class I major histocompatibility complex (MHC)-specific inhibition and prevents bone marrow allograft rejection. J. Exp. Med. 184:2037.

65. Zajac, A. J., R. E. Vance, W. Held, D. J. D. Sourdive, J. D. Altman, D. H. Raulet, and R. Ahmed. 1999. Impaired anti-viral T cell responses due to expression of the Ly49A inhibitory receptor. J. Immunol. 163:5526.

66. Sawai et al., AJRI 34:26-34 (1995).

67. Better et al., Science 240:1041-1043(1988)

68. Imani, F., and M. J. Soloski. 1991. Heat shock proteins can regulate expression of the TIa region-encoded class 1b molecule Qa-1. Proc. Natl. Acad. Sci. U.S.A. 88:10475.

69. Leclercq, G., J. Plum, D. Nandi, M. De Smedt, and J. P. Allison. 1993. Intrathymic differentiation of V gamma 3 T cells. J. Exp. Med. 178:309.

70. Van Beneden, K., F. Stevenaert, A. De Creus, V. DeBacker, J. De Boever, J. Plum, and G. Leclercq. 2001. Expression of Ly49E and CD94/NKG2 on fetal and adult NK cells. J. Immunol. 166:4302.

71. Havran, W. L., and J. P. Allison. 1990. Origin of Thy-1+dendritic epidermal cells of adult mice from fetal thymic precursors. Nature 344:68.

72. Leclercq, G., M. De Smedt, B. Tison, and J. Plum. 1990. Preferential proliferation of T cell receptor V gamma 3-positive cells in IL-2-stimulated fetal thymocytes. J. Immunol. 145:3992.

73. Gays, F., K. P. Fraser, J. A. Toomey, A. G. Diamond, M. M. Millrain, P. J. Dyson, and C. G. Brooks. 2001. Functional analysis of the molecular factors controlling Qa1-mediated protection of target cells from NK lysis. J. Immunol. 166:1601.

74. Aldrich, C. J., A. DeCloux, A. S. Woods, R. J. Cotter, M. J. Soloski, and J. Forman. 1994. Identification of a Tap-dependent leader peptide recognized by alloreactive T cells specific for a class 1b antigen. Cell 79:649.

75. Bai, A., J. Broen, and J. Forman. 1998. The pathway for processing leader-derived peptides that regulate the maturation and expression of Qa-1 b. Immunity 9.413.

76. Huston et al., Methods in Enzymology 203:46-88 (1991).

77. Shu et al., PNAS 90:7995-7999 (1993).

78. Skerra et al., Science 240:1038-1040 (1988)

79. Morrison, Science 229:1202 (1985)

80. Oi et al., BioTechniques 4:214 (1986)

81. Gillies et al., (1989) J. Immunol. Methods 125:191-202

82. Riechmann et al., Nature 332:323 (1988),

83. Padlan, Molecular Immunology 28(4/5):489-498 (1991)

84. Studnicka et al., ProteinEngineering 7(6):805-814 (1994);

85. Roguska. et al., PNAS 91:969-973 (1994)

86. Harlow et al., Antibodies: A Laboratory Manual, Cold Spring Harbor Laboratory Press, 2nd ed. (1988)

87. Hammerling, et al., in: Monoclonal Antibodies and T-Cell Hybridomas 563-681, Elsevier, N.Y., (1981)

88. Russell E. Vance, Amanda M. Jamieson, and David H. Raulet Recognition of the Class 1b Molecule Qa-1 b by Putative Activating Receptors CD94/NKG2C and CD94/NKG2E on Mouse Natural Killer Cells. J. Exp. Med. 1999; 190:1801-1812.

89. Jespers et al., Bio/technology 12:899-903(1988).

The references cited herein and above are incorporated in their entirety herein by reference.

1. A monoclonal antibody which specifically binds to the inhibitory receptor Ly49E or a fragment of said receptor.

2. A monoclonal antibody according to claim 1 which further specifically binds to receptor Ly49C or a fragment of said receptor.

3. A monoclonal antibody according to claim 1 or 2 wherein said receptor is present on a hematopoietic cell.

4. A monoclonal antibody according to claim 3 wherein said hematopoietic cell is a cell selected from the group consisting of a natural killer cell, a memory T cell, a natural killer T cell, a gamma/delta T cell and an alpha/beta T cell.

5. A monoclonal antibody according to claim 1 or 2 which is the antibody 4D12.

6. A monoclonal antibody which specifically binds to at least one receptor selected from the group consisting of a CD94/NKG2A receptor, a CD94f NKG2C receptor and a CD94/NKG2E receptor.

7. A monoclonal antibody according to claim 6 wherein said receptor is present on a hematopoietic cell.

8. A monoclonal antibody according to claim 7 wherein said hematopoietic cell is at least one cell selected from the group consisting of a natural killer cell, a memory T cell, a natural killer T cell, a gamma/delta T cell and an alpha/beta T cell.

9. A monoclonal antibody according to claim 7 or 8 which is the antibody 3S9.

10. A humanized or chimeric antibody comprising a fragment of a monoclonal antibody according to any of claims 1 to 9.

11. A fragment or a homologue of any of the monoclonal antibodies according to any of claims 1 to 10 which binds to said receptor or a fragment of said receptor.

12. The antibody according to any of claims 1 to 10 or a fragment or homologue thereof which further comprises a detectable label.

13. A hybridoma which produces a monoclonal antibody according to any of claims 1 to 9.

14. Method for the detection of at least one of a Ly49 or CD94/NKG2 receptor comprising contacting a monoclonal antibody of any one of claims 1 to 10 or a fragment or homologue thereof with a cell or sample suspected of expressing or containing said receptor and detecting the binding of said antibody with said cell expressing said receptor or a receptor of said sample.

15. Method according to claim 14 wherein said detection is performed by a fluorescent activated cell sorter (FACS), an enzyme linked immunosorbent assay (ELISA), a radioimmunoassay (RIA), a fluorescent immunoassay (FIA), an immuno radio metric assay (IRMA), a chemiluminescent immuno assay (CLIA), an electro chemiluminescent immuno assay (ECL), an agglutination assay, or a histological immuno assay.

16. A kit for the detection of a Ly49 or CD94/NKG2 receptor, comprising at least one monoclonal antibody or a fragment or homologue thereof according to any of claims 1 to 9, or a humanized antibody according to claim 10.

17. A pharmaceutical composition comprising a monoclonal antibody or a fragment or homologue thereof according to any of claims 1 to 9, or a humanized antibody according to claim 10, optionally in admixture with a pharmaceutically acceptable carrier.

18. A process for obtaining and isolating a hybridoma according to claim 13, comprising:
(a) providing immunized spleen cells of an animal, said animal having been previously immunized in vivo with an antigen expressing cells chosen from hematopoietic cells from rat or mice,
(b) fusing said immunized spleen cells with myeloma cells under hybridoma-forming conditions to form hybridomas, and,
(c) selecting hybridomas which secrete monoclonal antibodies which specifically bind to a Ly49 or a CD94/NKG2 receptor.

19. A monoclonal antibody according to any of claims 1 to 9 obtainable by a process comprising:
(a) providing immunized spleen cells of an animal, said animal having been previously immunized in vivo with an antigen expressing cells chosen from hematopoietic cells from rat or mice,
(b) fusing said immunized cells with myeloma cells under hybridoma-forming conditions,
(c) selecting those hybridomas which secrete monoclonal antibodies which specifically bind to a Ly49 or a CD94/NKG2 receptor,
(d) culturing the selected hybridomas of step (c) in a culture medium, and
(e) recovering monoclonal antibodies secreted by said selected hybridomas; or,
(f) implanting the selected hybridomas obtained in step c) into the peritoneum of a mouse and, when ascites has been produced by the animal, recovering the monoclonal antibodies then formed from said ascites.

20. A process for producing a monoclonal antibody according to any of claims 1 to 9 comprising:
(a) culturing ahybridoma which secretes said antibody in a culture medium,
(b) recovering the monoclonal antibodies secreted by said hybridomas; or,
(c) implanting the hybridomas into the peritoneum of a mouse and, when ascites has been produced by the animal, recovering the monoclonal antibodies then formed from said ascites.

21. A method for inhibiting or enhancing the cytolytic activity of cells expressing a receptor of the Ly49 or CD94/NKG2 family comprising administering or adding a monoclonal antibody according to any of claims 1 to 9 or a fragment or homologue thereof, or a humanized version thereof according to claim 10, to a mammalian recipient or to a cell culture or tissue culture.

22. A method for treating or preventing cell, tissue or organ transplant rejection in a mammalian recipient, comprising administering an antibody that binds to an Ly49 or CD94/NKG2 inhibitory receptor in an amount effective to inhibit an immune-mediated response in the recipient to said transplant, wherein said antibody is an antibody of any of claims 1 to 10 or a fragment or a homologue thereof.

23. The method according to claim 22 wherein the amount is effective to induce immune tolerance in the recipient to the transplant.

24. The method according to claim 22 wherein the cell, tissue or organ transplant is one of a pancreatic islet, a kidney, a heart, a vascular tissue, a hematopoietic cell or a liver.

25. The method according to claim 23 wherein the cell, tissue or organ transplant is one of a pancreatic islet, a kidney, a heart, a vascular tissue, a hematopoietic cell or a liver.

26. The method according to claims 22 wherein the recipient is human.

27. The method according to claim 23 wherein the recipient is human.

28. The method according to claim 24 wherein the recipient is human.

29. A method for reducing the incidence of acute rejection episodes following transplantation, comprising administering to human patients undergoing transplantation a therapeutically effective dosage of a chimeric or humanized monoclonal antibody according to claim 10 that binds to a Ly49 or CD94/NKG2 inhibitory receptor and inhibits binding of the cell comprising said receptor to the cells of the transplant.

30. A pharmaceutical composition comprising an effective immunosuppressive amount of an antibody which binds to a Ly49 or CD94/NKG2 inhibitory receptor in combination with a pharmaceutically acceptable vehicle.

31. The pharmaceutical composition of claim 30 wherein the amount is effective to induce immune tolerance in a mammal recipient of an allogeneic or xenogeneic organ, tissue or cell transplant.

32. A method for treating or preventing cell, tissue or organ transplant rejection in a mammalian recipient according to claim 22 or 29 further comprising administering at least one anti-inflammatory or immunosuppressive drug.

33. The method of claim 32 wherein said anti-inflammatory or immunosuppressive drug is cyclosporin, cyclophosphamide, FK-506, rapamycin, corticosteroids, mycophenolate mofetil, leflunomide, anti-lymphocyte globulin, deoxyspergualin or OKT.

34. A pharmaceutical composition according to claim 30 further comprising an anti-inflammatory or immunosuppressive drug.

35. A combination therapy for treating or preventing cell, tissue or organ transplant rejection in a mammalian recipient comprising administering an effective immunosuppressive amount of an antibody which binds to an Ly49 or CD94/NKG2 inhibitory receptorand an anti-inflammatory or immunosuppressive drug.

36. A method for treating or preventing autoimmune disease comprising administering an effective immunosuppressive amount of an antibody which binds to an Ly49 or CD94/NKG2 inhibitory receptor.

37. The method for treating or preventing autoimmune disease according to claim 34 wherein the autoimmune disease is inflammatory bowel disease, multiple sclerosis, Type I diabetes, systemic lupus erythematosus or rheumatoid arthritis.

38. A method of manufacturing a medicament comprising admixing a monoclonal antibody of any one of claims 1 to 10 or a fragment or a homologure thereof with a medicament diluent or carrier.
